(19) United States
(12) Patent Application Publication
(10) Pub. No.: US 2005/0089847 A1
(43) Pub. Date: Apr. 28, 2005

(54) Short chain dehydrogenases/reductases(sdr)

(76) Inventor: Thomas Wilckens, Munchens (DE)

Correspondence Address: SUTHERLAND ASBILL & BRENNAN LLP, 999 PEACHTREE STREET, N.E., ATLANTA, GA, 30309, US

(21) Appl. No.: 10/344,326

(22) PCT Filed: Aug. 7, 2001
(86) PCT No.: PCT/EP01/09140

Related U.S. Application Data
(60) Provisional application No. 60/223,436, filed on Aug. 7, 2000.

(51) Int. Cl. C12Q 1/68; C12Q 1/26

(52) U.S. Cl. 435/6; 435/25

(57) ABSTRACT

The present invention relates to a method for identifying or verifying members of the short chain dehydrogenase (SDR) family, to a method for providing modulators for members of the SDR family and to the preparation of pharmaceutical agents using these modulators.

The present invention relates to a method for identifying or verifying members of the short chain dehydrogenase (SDR) family, to identified SDRs, to a method for providing modulators for members of the SDR family and to the preparation of pharmaceutical agents using these modulators.

### TECHNICAL BACKGROUND

The short chain dehydrogenase/reductase (SDR) protein family (H. Jörnvall et al., Biochemistry 34 (1995), 6003-6013) is an old conserved protein family, the members of which show a residue identity level of only 20-30%. However, it has been found that the three-dimensional structure of members of the SDR family are highly similar, determining their functions and affiliation to the SDR family (U. Oppermann et al., Enzymology and Molecular Biology of Carbonyl Metabolism 6, Weiner et al. eds., Plenum Press, New York (1996), p. 403-415).

While initially only two structures of SDR enzymes restricted to bacterial and insect enzymes have been discovered, rapid progress on the knowledge of short chain dehydrogenases/reductases resulted in an increasing number of structures, which could be assigned to the SDR family. Currently, about 1.600 putative members are known, from which up to 100 may be derived from human, such as hydroxysteroid dehydrogenases (HSD).

An approach to identify SDR proteins is described in W. N. Grundy et al., Biochemical and Biophysical Research Communications 231 (1997) 760-766 and in T. L. Bailey et al., J. Steroid Biochem. Molec. Biol. 62 (1) (1997) 29-44. Therein homologies are searched for via a hidden Markov model, i.e. a self-training model, and thus classified to a certain protein family. A classification based on the function is not made in these models.

Since the SDR enzymes are involved in various metabolitic pathways and show different activities, such as oxidoreductases, lyases, or epimerases and, as discussed above, show only a low identity of 20-30%, it has been difficult, to assign new members unambiguously to the SDR family and to find modulators therefor.

However, since HSD and other SDR play a critical role in higher vertebrates, it is desirable to discover further members of the SDR family and establish modulators for known and new SDR enzymes.

It was therefore an object of the present invention to provide an algorithm which allows for the identification or verification of SDR family members with high confidence levels.

It was a further object of the invention to provide an algorithm which provides a search hierarchy with various levels.

It was another object of the present invention to provide modulators for SDR family members.

Still another object of the invention was to provide pharmaceutical agents based on members of the SDR family.

### SUMMARY OF THE INVENTION

The present invention relates to a method for identifying or verifying members of the short chain dehydrogenase (SDR) family based on an algorithm using core SDR motifs for searching members of the SDR family. Further, the present invention relates to a method for providing modulators for such members of the short chain dehydrogenase (SDR) family, which enhance or inhibit the activity therefrom as well as a method for providing a pharmaceutical agent using modulators for members of the SDR family.

In particular the present invention provides a combination of the steps (i) screening databases to search and find SDR sequences, (ii) store the data on an appropriate medium, rank and validate the hits and (iii) using the SDR sequences found to develop new drugs.

### DETAILED DESCRIPTION OF THE INVENTION

Members of the SDR protein family have a common core sequence, which is about 250-350, preferably about 260-290 and in particular about 270 amino acids in length. SDR proteins can have extensions at the N-terminus and/or at the C-terminus. Typically, these extensions have a length of 20 to several hundred, in particular up to 500 amino acids. These extensions can be membrane anchors or other signals or they can constitute completely distinct protein domains. Therefore, according to the invention it is primarily searched for SDR core domains, the rest of the protein being analysed only later on.

In a first embodiment the invention provides a method for identifying or verifying members of the short chain dehydrogenase (SDR) family comprising the steps
(a) providing a target sequence of molecules to be classified,
(b) comparing said target sequence with core SDR motifs selected from
   (i) MV1 being derived from the motif MT1:TGxxxGxG by replacement of 0 to 2 amino acids,
   (ii) MT2:NN(0-2:x)AG,
   (iii) MT3:N, located at a position 90-110 relative to MT1,
   (iv) MV4 being derived from the motif MT4:S(11-52:x)YxxxK by replacement of 0-2 amino acids and
   (v) MT5:PG,
(c) determining positive SDR candidates containing
   (i) at least the core SDR motifs MV1 and MV4 and
   (ii) at least 7 of the 14 amino acids contained in the motifs MT1, MT2, MT3, MT4 and MT5 and
(d) classifiying positive SDR candidates as belonging to the SDR family.

It has been found in many SDR proteins that several motifs of the SDR core domain often occur in combination. However, it is not obligatory that all SDR core motifs are present for a protein to be an SDR enzyme. Since SDR proteins may lack one or several of the core SDR motifs, they may not be found by simple comparison of the complete SDR core domains.

Within the SDR core the following functional motifs frequently are found. The motifs are given in order from N-terminus to C-terminus assigning a position number 0 to the start of the first motif MT1, which of course need not be the start of the complete SDR protein.
MT1:TGxxxGxG (circa position 0-7);
MT2:NNAG (circa position 75-78);
MT3:N (circa position 100);
MT4:S-Y-K (circa positions 128/142/146) and
MT5:PG (circa position 170/171).

Using these motifs, the algorithm according to the invention has been developed, which allows for an assignment of target sequences to be an SDR sequence with a confidence level of more than 95%, in particular ore than 98%. By relying on motifs of the core SDR region positive hits due to indentity in non significant regions can be excluded. It is essential for the present invention that the core SDR motifs were selected because of their functional meaning and not only because of homology comparisions. The SDR motifs used form essential parts of nucleotide co-factor binding region (Rossman-fold) and the active site of members of the SDR family. The motifs MT1 and MT2 represent components of the co-factor binding site. A particular co-factor of SDR enzymes is NAD(P)(H). The motif MT3 represents a contact to the active site and the motif MT4 a part of the active site. The motif MT5 is of functional importance due to its proximity to the co-factor. Thus core SDR motifs are motifs which are essential for the functionality of the SDRs.

For detecting members of the short chain dehydrogenase (SDR) family in the method according to the invention it is therefore essential that functional aspects are considered, wherein enzymatically active SDRs are detected and not only sequences which exhibit a certain homology to other SDRs at functionally irrelevant positions.

Contrary to prior art algorithms, according to the invention those amino acids are taken into account which are essential for the function. A minimum amount of the amino acids selected thus enables a maximum amount of targets due to the divergence of the SDR family, wherein the detection of erroneously positive targets is basically excluded because of the connection between function and structure. This way the target specificity can be considerably improved over algorithms, such as neuronal networks, which are based on homology comparisons (cf. J. A Gerlt et al., Genome Biology, 1 (5) (2000), Reviews 0005.1-0005.10). In addition, further functional information can be easily included in order to screen for functional deficits, such as screening for an associated disease mutations or individualized drug metabolism.

While the individual proteins assigned to the SDR family using the algorithm of the invention may have identities of only 30% or less, they show a very similar three-dimensional structure. It is important for the correct formation of the desired three-dimensional SDR structure that motifs 1 to 5 are present in the above listed succession.

For the description of the motifs the single letter amino acid code is used. x denotes a variable amino acid, selected preferably from the 20 naturally occuring amino acids. NN(0-2:x)AG means that 0, 1 or 2 amino acids can be positioned between amino acids N and A. S(11-52:x)YxxxK means that from 11 to 52 amino acids are positioned between S and Y and 3 amino acids are positioned between Y and K.

A replacement of 0-2 amino acids refers to a replacement of any of the amino acids given (including x), whereby preferably the explicitly named amino acids are replaced. A replacement includes deletion of the amino acid or a substitution of the amino acid by another amino acid selected preferably from the 20 naturally occuring amino acids. The replacement of 1 or 2 amino acids results in a fuzzy logic including also sequences, in which the motifs are not 100% conserved. A strategy combining sequence and structure information is also disclosed by L. Yu et al., Protein Science 7 (1998), 2499-2510.

In a preferred embodiment of the invention MT2 is defined to be NNAG (i.e. without any amino acids x between NN and AG), but with possible replacement of 1-3 amino acids.

The motif MT3:N is located at position 90-110, preferably at position 95-105 and in particular at position 100 relative to the start of the motif MT1.

In a particularly preferred embodiment of the invention the second part of motif MT4 is defined to be the pattern YxASK with possible replacement of up to 3 of these residues. In this preferred embodiment the range of possible scores is extended from 0-14 up to 0-16. In this embodiment positive candidates have a score of at least 7, preferably at least 9, more preferably at least 11 and most preferably at least 13.

Preferably the SDR motifs are located in the order given from the N-terminus to the C-terminus for a sequence to be classified as SDR sequence. The positions given in brackets above may be shifted by amino acid insertions or deletions within the sequence analyzed. Preferably the motifs are found within ±50, more preferably ±20 positions, in particular ±10 positions and most preferably ±5 positions, from the values given.

A target sequence is classified as belonging to the SDR family according to the invention, if it contains at least the core SDR motifs MV1 and MV4 and at least 7 of the 14 explicitly named amino acids contained in the motifs MT1, MT2, MT3, MT4 and MT5. The confidence level can be controlled by varying the amount of matching amino acids, which have to be present in the target sequence. Therefore, if a high confidence level, e.g. >98%, more preferably >99% is desired, it may be preferable to classify target sequences as positive SDR candidates, only if they contain at least 9 of the 14 amino acids, or even at least 11 or at least 12 of the 14 amino acids contained in the motifs MT1-MT5. Setting the score at a value of at least 13 results in the detection of exclusively sequences, which are an SDR with a confidence level of almost 100%, e.g. >99.8%.

In a preferred implementation of the method of the invention a file is provided containing a set of protein amino acid sequences, the input set. One sequence is taken from the input set, the query sequence. The implementation then passes the query sequence to the algorithm, which examines it for occurences of some or all of MT1-5 in the arrangements allowed. The algorithm returns a list of the best possible combinations of occurences. If the matches contain more than a specified number of amino acids from MT1-5, they are assigned as hits.

In a particularly preferred embodiment the method of the invention is as follows:

The algorithm first searches the whole sequence for instances of the first motif MT1, allowing for up to two replacements as described. Each possible MT1 match is then taken as the origin for searches for the motifs MT2 to MT5 whose positions are defined relative to the position of MT1. A data structure based on each position of MT1 is created, which will be used to store the positions of other motifs relative to this MT1.

For a given MT1 match at position P, the preferred position of the motif MT2 is P+75. According to the rules MT2 is preferably at position (P+75)+/−50, more preferably (P+75)+/−20. This defines a window on the sequence within which instances of the motif MT2 are searched for, including any variants of MT2 with up to three replacements. Since the size of the window affects the time taken to search and the quality of the matches found, the preferred implementation allows the window sizes to be specified for each search. Any possible matches within the window are added to the result data structure as children of the current MT1.

The procedure is then repeated for instances of MT3, where the window is (P+100)+/−50, more preferably (P+100)+/−20, or any other specified window size. Again, any results found within the allowed window are added to the result data structure as children of the current MT1.

The same procedure is then followed for MT4, with a window (P+128)+/−50, more preferably (P+128)+/−20, or any other specified window size. In this case the window only specifies the position of the Serine residue of MT4, and once a candidate Serine has been found at position P_{S} (and added to the result structure as a child of the current MT1), it defines a window P_{S}+(11-52), within which instances of the second part of MT4 are searched for, allowing for replacements. Any candidates are added to the result structure as children of the current Serine match.

Since MT4 allows replacements, and those replacements could include replacements of the Serine, the implementation additonally searches for the second part for MT4 in cases where the Serine is not found. In this case, a virtual window composed of all of the possible positions of the (missing) Serine, offset by the P_{S}+(11-52), is constructed [P_{S}+(11-52)+/−20, i.e. the range P+128+(11-20) to P+128+(52+20)], or likewise for any specified window size. If any instances of the second part are found they are added to the result data structure as children of the current MT1.

The procedure is then repeated for instances of MT5, where the window is (P+170)+/−50, more preferably (P+170)+/−20, or any other specified window size. Again, any results found within the allowed window are added to the result data structure as children of the current MT1.

At this stage the implementation holds in memory a tree-structured data structure where the possible matches with the specified pattern correspond to depth-first traversals of the tree. The implementation enumerates the possible combinations of the full or partial motifs, adds up a score calculated from the number of residues in the motifs which were actually matched, and discards the instances where the overlapping windows have given rise to motifs where the ordering is not MT1-MT2-MT3-MT4-MT5. Any combination with a score equal to the maximum score found is kept and added to a list, and it is this list with its score, the motifs found, and the position in the sequence of each amino acid matched which is returned as the result at this stage of the implementation.

The preferred implementation includes significant enhancements, in particular:
MT2 is defined to be NNAG without the presence of 1 or 2 amino acid insertions between the NN and AG parts. The implemenation allows for replacement of 1-3 of the residues, and will continue to search for other motifs even if no instance of MT2 is found.
The second part of MT4 is defined to be the pattern Y*ASK instead of Y**K, but again the implementation allows replacement of up to 3 of these residues. This makes the range of possible scores 0-16 instead of 0-14.
The absence of motif MT4 is not used to discard SDR candidates, but the effect on the overall score of its absence (5 out of a possible 16 matches) is significant in excluding matches which do not contain it and additionally the presence of the active site MT4 tyrosine is indicated for each result, as a significant indicator of possible SDR catalytic activity.

In a further preferred embodiment an enlargement or optimization, of the algorithm is performed also on human extended SDRs. Thus it is taken into account that compared to the other SDRs often only a motif MTₓ1 (TGxxGxxG) as well as a motif MTₓ4 (YxxxK) is present, wherein MTₓ1 is a variant of TGxxxGxG. For determining human extended SDRs with this enlarged algorithm motifs 2, 3 and 5 can even be missing.

In a particularly preferred embodiment the algorithm according to the invention comprises the import of a data set, e.g. from data bases, organizing the data set by using the method according to the invention, ranking the SDR hits and further analyzing and managing the data of the detected hits, such as a cross-linking to data bases, to BLAST or to other tools.

Subject matter of the invention is also a data carrier, particularly a diskette containing the method according to the invention and particularly the above described algorithm.

Whereas the method according to the invention itself already has a very high specificity and reliability in the selection of SDR candidates, the SDR candidates detected can be subjected to further evaluation criteria. These criteria are e.g. comparing the 3D-structure of the candidates detected with the 3D-structure of known SDR proteins or a standardized 3D-structure, which is derived from SDR candidates identified by the method according to the invention. Thus, in a further preferred embodiment of the invention the polypeptides classified as positive SDR candidates in the method according to the invention are subjected to another evaluation step in view of their three-dimensional structure in order to further improve the selectivity and specificity of the method. Thus it is possible to use known three-dimensional structures of SDR family members (cf. e.g. H. Jörnvall, Biochemistry 34 (1995), 6003-6013; U. Oppermann et al., Adv. Exp. Meth. Biol. 414 (1997), 403-415 or J. Benach et al., J. Mol. Biol. 282 (1998), 383-399). However, it is also possible to determine the three-dimensional structures of the SDR candidates detected with the method according to the invention and to prepare a common comparative three-dimensional structure therefrom. This way it can be examined, e.g. whether the positive SDR candidates exhibit the co-factor binding site typical of SDRs. A further criteria may be the presence of amino acid Y at position 152±20, particularly ±10. Further, it is possible to compare the amino acids sequences detected with known SDR sequences, e.g. via an alignment.

After the sequences have been classified as SDRs it is also possible to search for further domains, e.g. membrane domains in order to thus classify them to a certain type of tissue.

An important subgroup of SDRs are FabGs, which are derived from pathogens and which can be identified via the method according to the invention. Since FabGs are often strongly degenerated and thus exhibit a relatively low score (e.g. 9 or more) in the method according to the invention, it can be advantageous to examine possible FabG-SDR candidates in a second step in view of the presence of the following motif variations: MT_{y}2:VxVNNAG, wherein V can be replaced particularly by I, as well as MT_{y}5:PGFI, wherein F and/or I can be missing.

A list of FabG proteins which were identified by the method according to the invention is shown in Table 4. FabGs are involved in the lipid metabolism of bacteria and are particularly suitable for the development of antibiotics.

A further group of SDRs which can be identified by the method according to the invention are bacterial SDRs. Bacterial SDRs detected with the algorithm according to the invention are shown in Table 3.

Further, it is possible to detect production enzymes as well as thermostable enzymes with the method according to the invention.

In a most preferred embodiment, the so-called SDR Finder, the method according to the invention is based on the implementation of functional data both on the three-dimensional structure and on the biological function (NADP(H)-dependend enzymes). The implementation is hierarchically structured according to the smallest common denominator having a functional meaning. Contrary to known tools not motifs, but SDR candidates are searched for and thus also for those having a very low homology or hardly conserved core motifs, respectively. The search for SDR candidates according to the invention enables a considerably higher specificity. The SDR candidates detected are of biologically functional relevance. At the same time a greater number of hits is found due to the use of the smallest common denominator.

Further, it is possible to establish a ranking with the algorithm according to the invention, to export the data in different formats and to selectively search for species. Thus, the SDR_Finder represents an “all-in-one” analysis solution including various obtainable possibilites, particularly the woldwide web. The implementation of hyperlinks to NCBI, EMBL and their tools (e.g. Blast, ClustalW, PfaM, PDB, Medline, OMIN) represents an “in silico” analysis/drug development software of modular structure which is particularly developed for SDR. Further modules which can be connected thereto are the examination of three-dimensional structures, the determination of active centres and the substrate docking simulation. The latter can also be implemented directly into the SDR_Finder and allow direct access, e.g. to 3D-databases and chemical libraries via the worldwide web.

In a preferred embodiment the SDR_Finder is equipped with fuzzy logic.

In addition, experimental data can be used, e.g. to evaluate the exchange of one amino acid in a motif regarding the functional consequences. This is of importance both for the individual adjustment of therapies and the evaluation of pathological problems or for the development of diagnostics, respectively.

Moreover, it is possible to enlarge the algorithm subgroup-specifically, as is shown herein for the FabG SDRs.

The method according to the invention can be used to verify sequences, which are already classified as (putative) SDR sequences, e.g. by automatic alignment (BLAST), to belong to the SDR family or not. Further, it can be used to search for and find new members of the SDR family or to search for and find new isoforms of SDR proteins. Therefore, the method of the invention provides additional information with regard to known sequences as well as to novel sequences. From the knowledge that a target sequence belongs to the SDR family as well as from the information obtained from the ranking findings about substrates and functions can be obtained. An important selection criteria thereby is the drugability of the SDR candidates detected.

The method according according to the invention can be used to detect e.g. human SDRs (human extended SDRs), animal SDRs, particularly mammalian SDRs, but also bacterial SDRs, FabG SDRs, fungi SDRs, SDRs of pathogens, SDRs of parasites, e.g. plant parasites.

The SDR proteins classified with the algorithm according to the invention thus can serve as platform for novel drug development. Human SDR proteins can particularly serve as starting point for the treatment of diseases or malfunctions of the body, whereas bacterial SDRs particularly provide a starting point for the development of novel antibiotics. Further, respecticve SDRs can serve for the development of antimicotica, pesticides, herbicides etc. . . .

While the algorithm of the present invention preferably is used to search for protein sequences, it is also possible the convert the motifs given into nucleic acid sequences and screen nucleic acid databases. A method to convert amino acid sequences into nucleic acid sequences while considering the degeneration of the genetic code is e.g. given from H. Jörnvall, FEBS Letters 456 (1999), 85-88. A search on the nucleic acid level can preferably be used to preselect sequences, which are then confirmed by an alignment in the protein level.

For the search on nucleic acid level these protein sequences are preferably converted to DNA sequences in particular cDNA sequences and used for the detection of further SDR candidates via a fuzzy logic or a hidden Markov model or via neuronal networks.

The method of the invention therefore also provides a tool for preselection of SDR candidates on the genomic level.

Preferably a ranking of the positive SDR candidate is performed e.g. according to the number of amino acids matching with motifs MT1-MT5. This way a hierarchy and/or an evolutionary relationship of the obtained SDR candidates can be obtained.

In a particularly preferred embodiment the target sequences classified as positive SDR candidates contain at least the core SDR motifs MT1 and MT4.

By hierarchically classifying the verification of the individual core SDR motifs several levels to detect SDR proteins can be obtained.

By using the algorithm according to the invention the search for SDR candidates and consequently the development of pharmaceuticals can be decisively enhanced. So far for the production of pharmaceuticals in vitro tissue cultures were admixed with different substrates. From cultures, wherein a certain substrate was converted, the target protein was isolated. According to the invention, this step and thus the knowledge of a substrate for the development of inhibitors or for the development of pharmaceuticals is not necessary. Moreover, starting from the sequence found a modulator, in particular an inhibitor or activator can be derived. This modulator can e.g. be derived from known modulators of other, in particular of related SDR proteins, suitable substrates, related functions and tissue distribution for 17β HSD isoforms are described e.g. by H. Peltoketo et al., J. Molecular Endocrinology 23 (1999), 1-11. Further, it is possible to derive a modulator from the 3D-structure of the SDR sequence. Such a 3D-structure can be obtained experimentally, e.g. by X-ray chrystallography or by computer based calculations, e.g. ab initio, force field, or rule based methods. Further, by inhibiting the active site of the SDR protein the function thereof can be determined.

The searching for SDR family members and ranking is also applicable to evaluate lead-candidates for possible inhibitors or modifiers of a specific enzyme. Leads may be derived from metabolites of evolutionary closely related or very distant enzymes from other species, if the same metabolite may not be found in the respective target organism. The evolutionary relationship of SDRs and their distinction from MDRs (medium chain dehydrogenase) is e.g. described by H. Jörnvall et al., FEBS Letters 445 (1999), 261-264 and AKRs (T. M. Penning, Endocrine Rev. 18(3) (1997) 281-305).

SDR enzymes are often involved in intermediary metabolisms, as well as in hormone and mediator metabolisms.

Substrates of known SDR proteins include e.g. steroids, such as estrone/estradiol, cortisone/cortisol and testosterone/3a-androstenediol. Thus, after classifying a sequence as SDR sequence functional tests for steroid substrates result in higher hit rates.

Further substrates of SDR proteins are UDP-glucose, UDP-N-actetylglucosamine, sepiapterin, dihydropteridine, R-3-OH-butyrate, dienoyl CoA, trans-Enoyl CoA, fatty acids, L-3-OH-acyl CoA. These substrates are particularly converted of SDR enzymes, which are involved in the intermediary metabolism. Further substrates of SDR proteins, particularly of SDR enyzmes, which are involved in hormone, mediator and xenobiotic metabolisms, are several hydroxy steroids, e.g. 3-beta-hydroxysteroids, 11-beta-hydroxy steroids or 17-beta-hydroxy steroids as well as prostaglandines and retinoides.

Further, searching SDRs, ranking and comparing evolutionary patterns can also be used to detect clinically relevant polymorphisms and/or single nucleotide polymorphisms (SNPs). This approach can be used to characterize diseased mechanisms as well as metabolism of xenobiotics, e.g. drug metabolism.

The identification of SDR members, ranking and comparing evolutionary patterns also allows for the identification of structure-function relationships. These structure-function relationsships are a key for identification of substrates of ORFs with unknown functions.

Within a lead oriented characterization first binding of a positive SDR candidate is evaluated. Starting from the binding a modulator, e.g. an inhibitor or activator, can be developed. Useful information for developing an inhibitor can be obtained from protein sequence alignment of full-length sequences, e.g. by comparison with known SDRs. Further, valuable information can be obtained from expressed sequence tags (EST) and gene sequence comparison. The procedure using the algorithm according to the invention allows for a great reduction of possible modulator candidates to be analysed and practically excludes target sequences, which are not SDR sequences. Therefore, an analysis of the functions in vitro or in vivo can be performed with much less effort than in the state of the art due to the reduced number of compounds to be tested. While in the methods according of the state of the art often the substrate must be known, this knowledge is not essential for developing modulators or/and drugs according to the invention. It is even possible to derive possible substrates in a subsequent step from the functions of the SDR enzymes found according to the invention. Ligands can be derived according to the procedure described by G. R. Lenz et al., DDT, 5(4) (2000), 145-156.

The validation of the potential SDRs found according to the algorithm of the invention, which can be used as new targets for drug development, can then be performed by experimental biochemical methods, such as high-throughput function screening for function identification, ultra high-throughput screening for lead compounds, transfection assays, knock out experiments, microarrays, tissue expression, cDNA arrays or analysis of disease in animal or in vitro model systems. However, it is also possible to use virtual methods using e.g. computers for validation of the new targets, e.g. by molecular homology modelling or substrate docking simulations.

Suitable strategies include e.g. gene expression of an identified SDR protein to obtain the protein molecule and subsequently performing biological functional assays and observe the behaviour of the cell.

Alternatively, the 3D-structure may be derived from the SDR sequence and an inhibitor for the active site provided. Using the inhibitor the function of the SDR within an organism can be evaluated.

Small weight inhibitors for SDR enzymes, which can be used as starting point for developing new or modified inhibitors, in particular inhibitors for newly identified SDR enzymes include:

1) Steroidal-based inhibitors like steroid carboxylates, acrylates, enolates 3,4- and 16,17-fused ring pyrazoles, 3 alpha, 17-beta or 20-beta-spiro-oxiranes as well as steroidal spirolactones, progestins, ursodexycholate, synthetic analogs of estrone sulfate and estrone-3-amino derivatives.

2) Inhibitors based on flavonoides and dihydropterin derivatives.

3) Inhibitors based on polyphenols and derivatives of 2,3-dihydroxy-1-naphthoic acids likegossypol (1,1′,6,6′,7,7′-hexahydroxy-5-5′-diisopropyl-3,3′-dimethyl-2,2′-binaphthalene-8,8′-dicarbaldehyde).

4) Inhibitors based on glycyrrhizin (3beta,20beta)-29-hydroxy-11,29-dioxoolean-12-en-3-yl 2-O-beta-D glucopyranuronosyl-alpha-D-glucopyranosiduronic acid) and components of enzymatically hydrolysed licorice extract like 3-O-beta-D-glucoronopyranosyl-24-hydroxy-18beta-glycyrrhetinic acid, 3-O-beta-D-glucur-onopyranosyl-18beta-glycyrrhetinic acid and 3-O-beta-D-glucuronopyranosyl-18beta-liquiritic acid, monoglycosylated derivatives of glycyrrhizin as well as carbenoxolone.

5) Pharmaceutically acceptable salts of the above mentioned molecules such as alkali metal (e.g. sodium), alkaline earth metal (e.g. magnesium) or ammonium as well as salts of organic carboxylic acids, such as acetic, citric, oxalic, lactic, tartaric, malic, isothionic, lactobionic, ascorbic and succinic acids; organic sulfonic acids, such as methanesulfonic, ethanesulfonic, benzenesulfonic and p-tolysulfonic acids; and inorganic acids, such as hydrochloric, sulfuric, phosphoric, and sulfamic acids.

Further candidates for inhibitors are chalcones (cf. Life Sci 68 (7) (2001) 751-761) as well as phytoestrogens (cf. Life Sci 66 (14) (2000) 1281-1291) and frenolicin and its derivatives.

Further, inhibitors can be derived from 3D-structures of the SDRs found, confirmed; identified or verified with the method of this invention, as is described e.g. by Liao et al., Structure, Vol. 9 (2001) 19-27.

Since SDR enzymes, in particular human SDR enzymes have been found to be involved in many pathways of the body, they are outstanding targets for developing new drugs. In particular human SDR enzymes have been found to be involved in intermediary metabolism, lipid mediator/hormone metabolism or xenobiotic phase I metabolism. On the other hand, SDR enzymes often constitute pathogenic factors causing diseases. Thus, e.g. the AME syndrome is associated with 11β HSD-2, bile acid metabolism is associated with 3β HSD, polycystic kidney disease is associated with Ke6(17β HSD-8) and Alzheimer's disease is associated with ERAB(17-β HSD-10).

Further diseases which can be effected by influencing, modulating or inhibiting SDRs comprise e.g. DHPR deficiency, phenylketonuria, dienoyl CoA reductase deficiency, galactosemia III, tetrahydrobiopterine deficiency, adrenal hyperplasia, adrenogenital syndrome, 11-oxoreductase deficiency, apparent mineralocorticoid excess syndrome, ovarian/breast cancer, male pyseudohermophroditism, Zellweger syndrome, pregnancy/ovarian cancer, polycystic kidney disease, Alzheimer's disease, retinits punctata albescens, retinitis pigmentosa, Down's syndrome, arterial hypertension, oncogenes, follicuolar lymphoma, hepatocarcinogenesis, aging related hormone deficiencies and immunity in general.

Since many of the SDR enzyme are bidirectional (reversible oxidoreaction) depending on the environment, it is also possible to provide a means for selectively enhance one of the enzymatic reaction, i.e. oxidation or reduction or to reverse the action observed.

Thus, providing new SDR sequences and modulators therefor, as described above, allows for the preparation of drugs or pharmaceutical agents, which can be used to control many different diseases. In particular drugs for treatment of cancer, e.g. breast cancer or prostate cancer, obesity, diabetes, fertility, osteoporosis, glucose metabolism, or conditions related to aging can be prepared. Further applications include steroid resistance, in particular ostrogen resistance and glucocorticoid resistance.

Further, SDR proteins and in particular hydroxy steroid dehydrogenases (HSDs) are outstanding targets for tissue-specific modulation of hormone-dependent or sensitive diseases, e.g. cancer, in particular prostate or breast cancer.

The present invention is in particular useful for providing a pharmaceutical agent for affecting immune regulation is provided by developing a modulator for 17β HSD type 3, 17β HSD type 7, 17β HSD type 8, 17β HSD type 10, 11β HSD-1, CR1, UDP glucose epimerase, SDR_SRL, AF067174, AF151840, AF151844, AF0078850, Fvt-1, HEP-27, DKFZ_ORF, WWOX_ORF, or CR3, a pharmaceutical agent for affecting autoimmunity is provided by developing a modulator for 17β HSD-3, 17β HSD-8, 11β HSD-1, AF057034, U89717, CR1, AF0078850, HEP-27, or CR-3, a pharmaceutical agent for wound healing or partial recovery is provided by developing a modulator for 17β HSD-3, 171 HSD-8, 11β HSD-1, U89717, CR1, AF0078850, HEP-27, or CR-3, a pharmaceutical agent for treatment of leukemia is provided by developing modulators for 17-β HSD-10 or Fvt-1 or a pharmaceutical agent for apoptosis regulation is provided by developing a modulator for 17β HSD-10, U89717, SDR_SRL; or for providing a pharmaceutical agent for affecting immune response by providing a modulator for AF016509, or providing a pharmaceutical agent for the treatement of cancer by providing modulators for AF016509, or providing a pharmaceutial agent for affecting cell growth by providing a modulator for U89717, or providing a pharmaceutical agent for the treatment of lung carcinoma by providing a modulator for SDR_SRL, or providing a pharmaceutical agent for the regulation of inflammation or vasculitis by providing a modulator for DKFZ_ORF.

The SDR candidates detected according to the invention can be used particularly for the production of inhibitors, such as antibodies on protein level or antisense on nucleic acid level. Moreover, it is possible to provide diagnostica by using the SDR candidates detected according to the invention, e.g. in order to show a malfunction.

An important aspect of the present invention in view of the development of new drugs for the diagnosis and/or treatment of a disease is that the inventive approach aims on a target family, i.e. SDRs and not on a specific disease. This allows for the development of a number of drugs, which all influence the same target family. By this approach the amount of experiments, effort and money necessary to develop a new drug can be significantly reduced, since many results can be used parallel for further members of the same target family leading to further new drugs for different medical applications. Further this approach allows for affecting a target which is known or suspected to be highly relevant for a person's health. In contrast to the classical approach wherein starting from a disease a suitable target must identified, this time and effort consuming procedure is not necessary with the inventive approach.

The invention is further elucidated by the following figures wherein

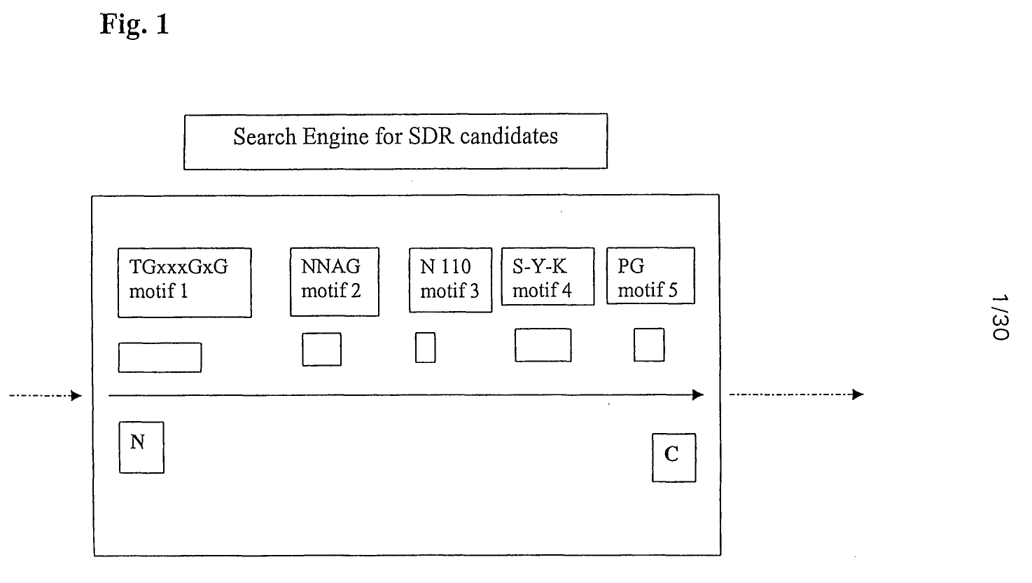

FIG. 1 represents the search engine for SDR candiates; The target sequence is compared to the specified core SDR motif, preferably in order from the N-terminus to the C-terminus.

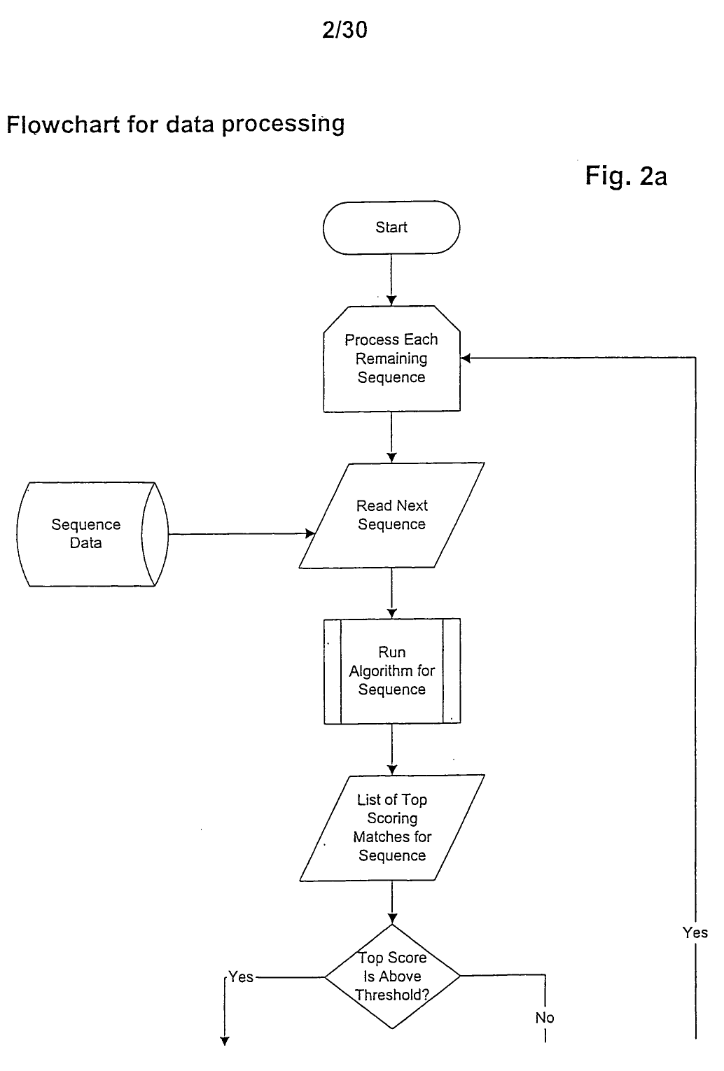

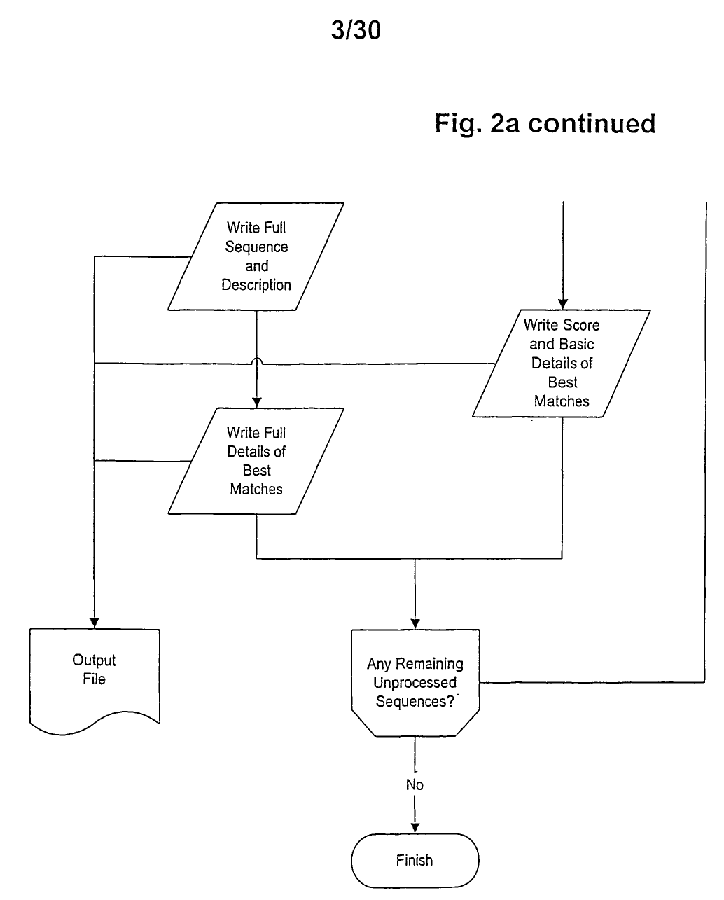

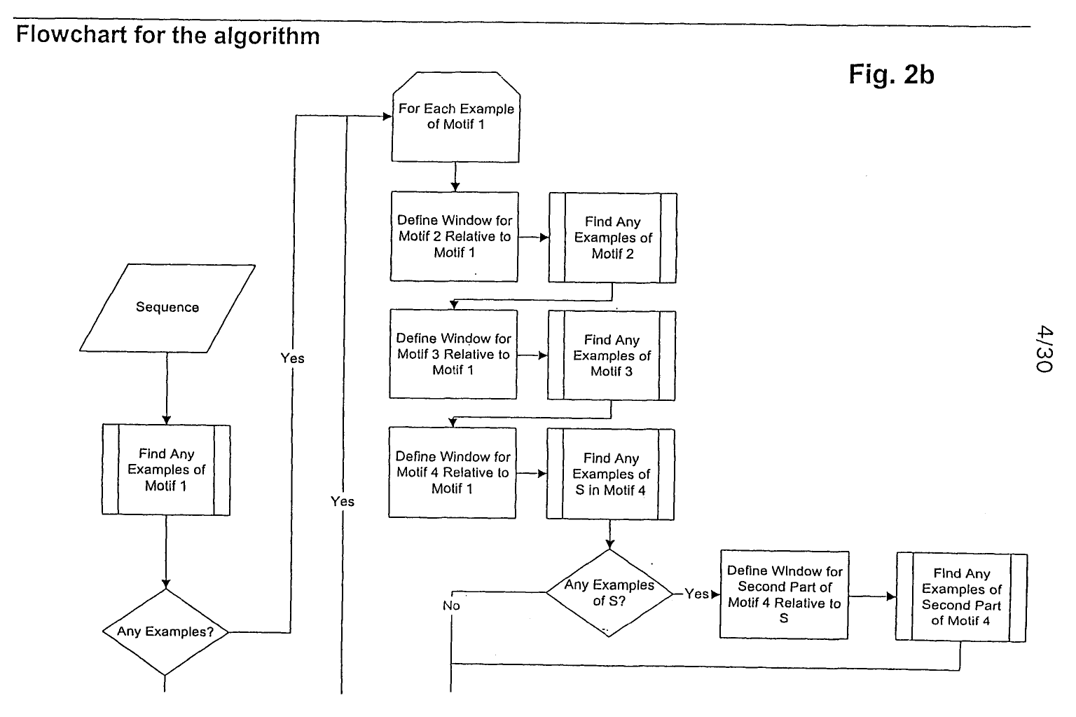

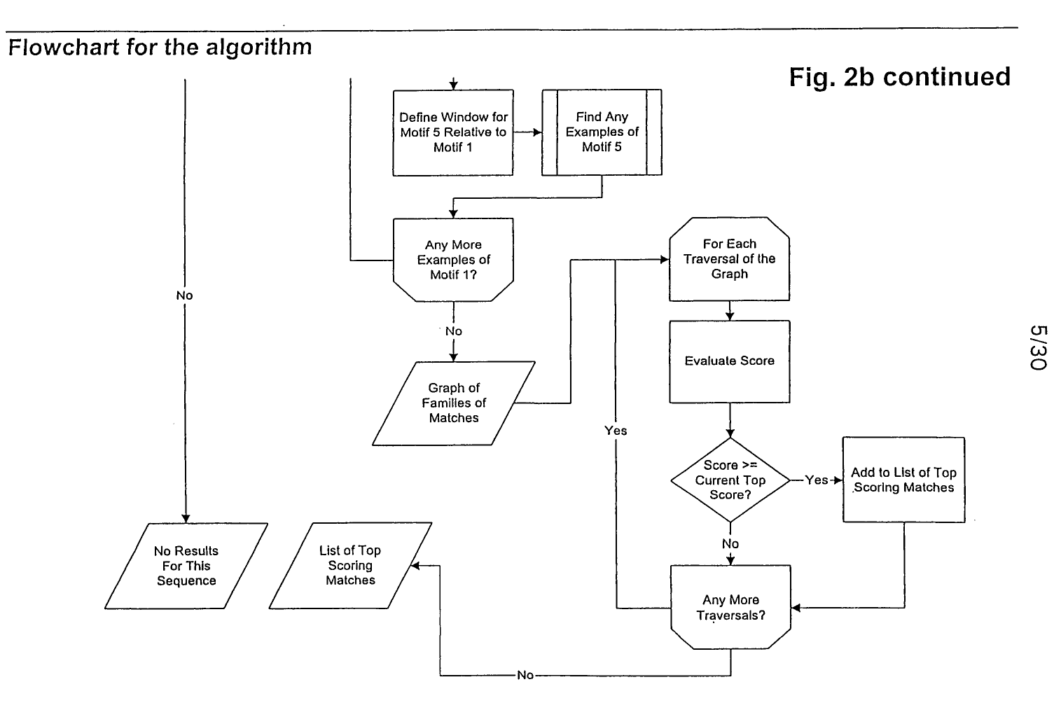

FIG. 2 shows flow charts for the preferred implementation of the algorithm. FIG. 2a shows a flow chart for data processing, while FIG. 2b shows a flow chart for the algorithm.

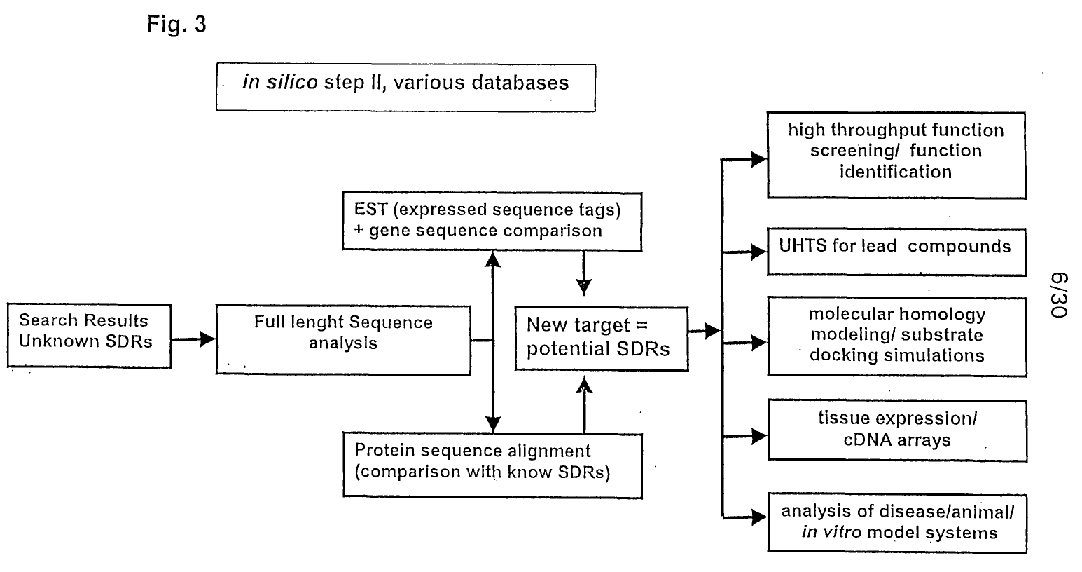

FIG. 3 depicts the development of pharmaceuticals on the basis of the SDR search according to the invention; The combination of virtual screening and classifying sequences to belong to the SDR family with the development of new drugs, as provided herein, is an efficient novel drug development strategy. By using the search results of the virtual SDR search new targets are obtained, from which drugs can be derived by various procedures.

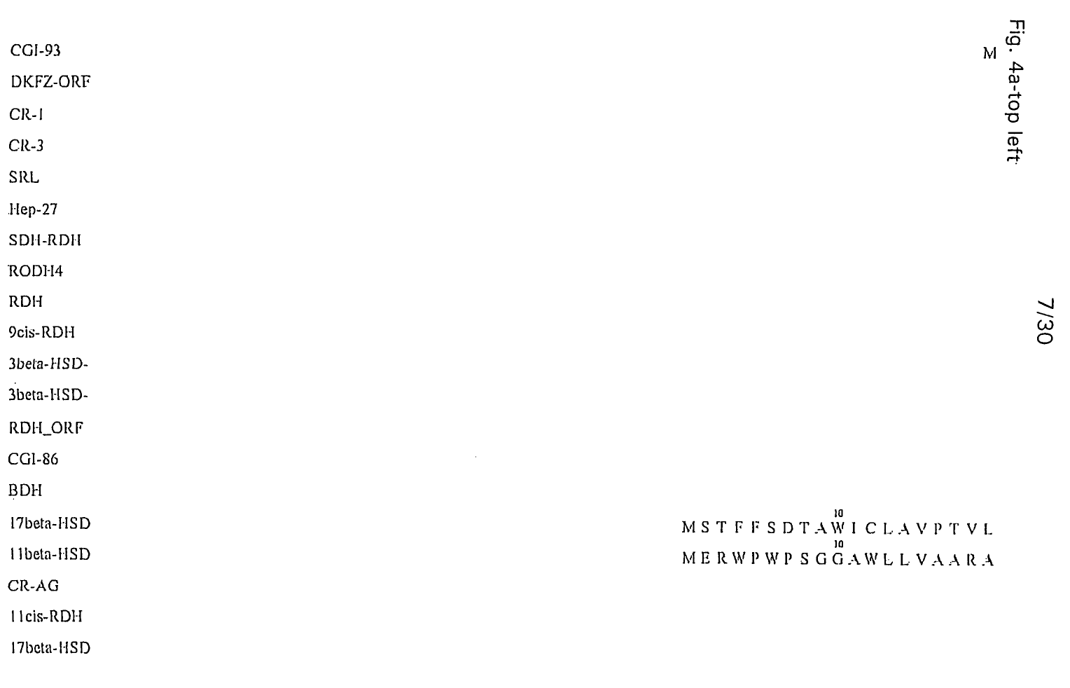

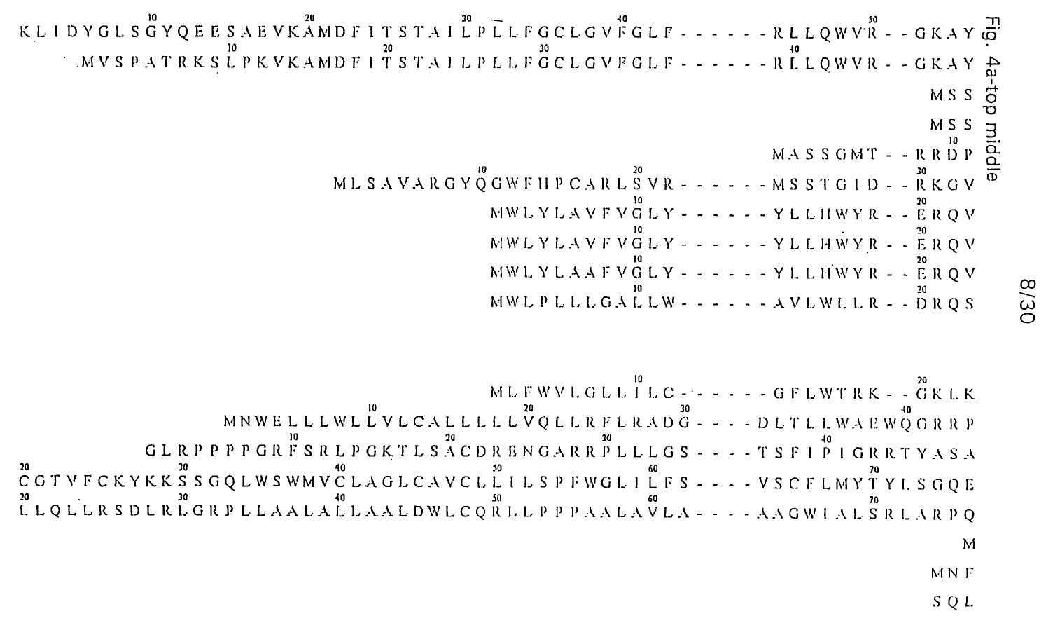

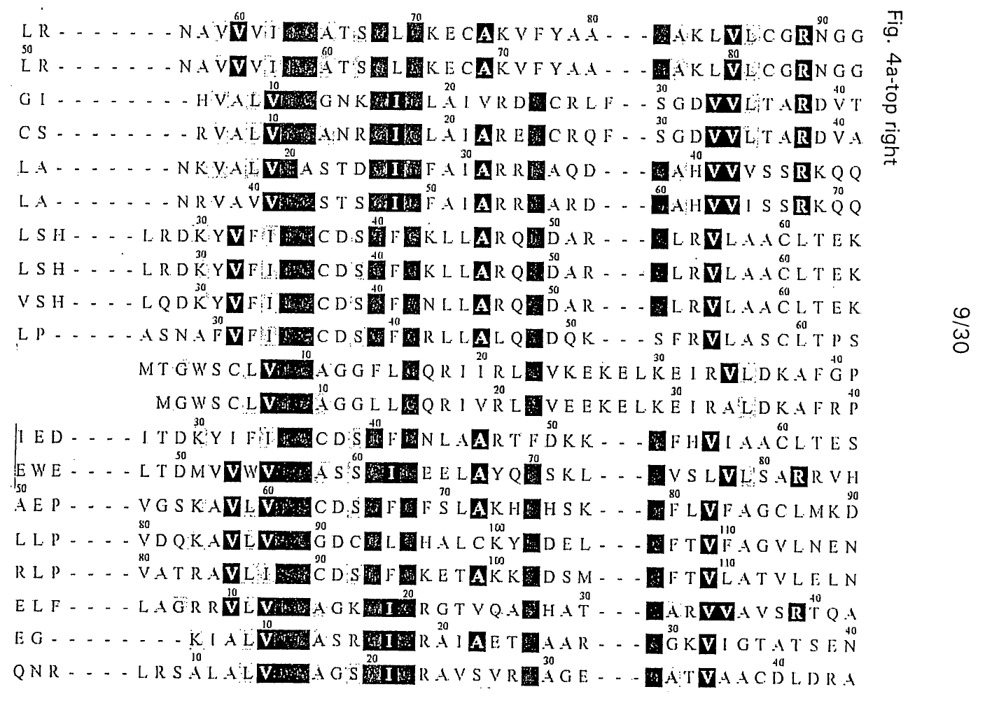

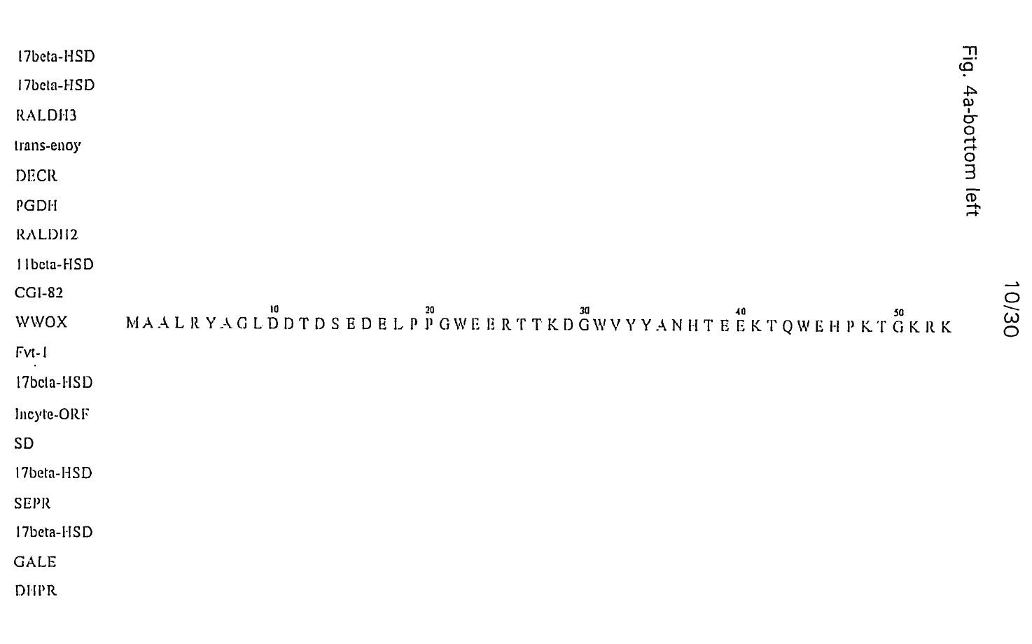

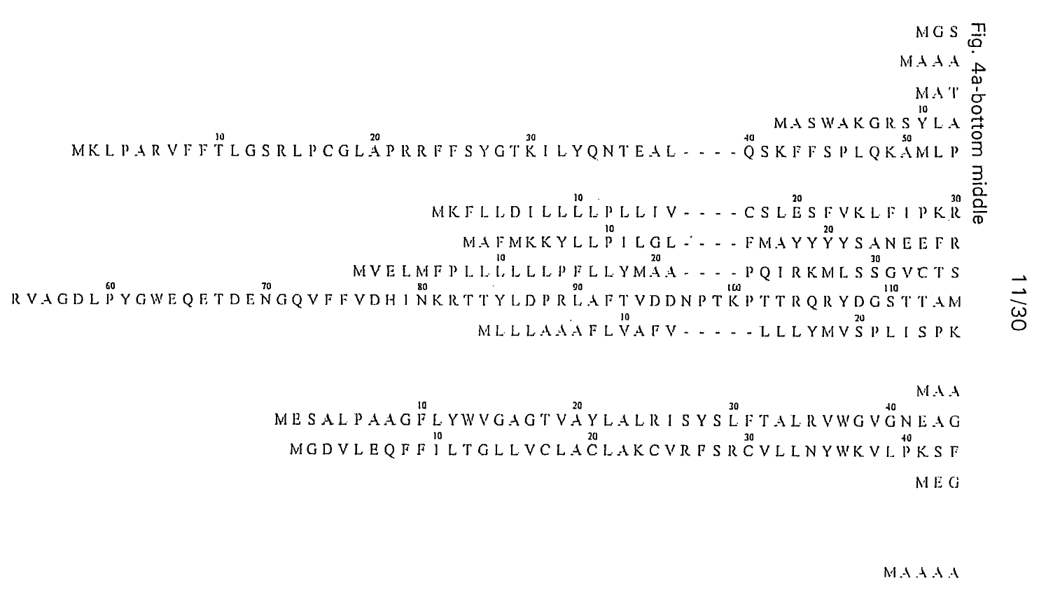

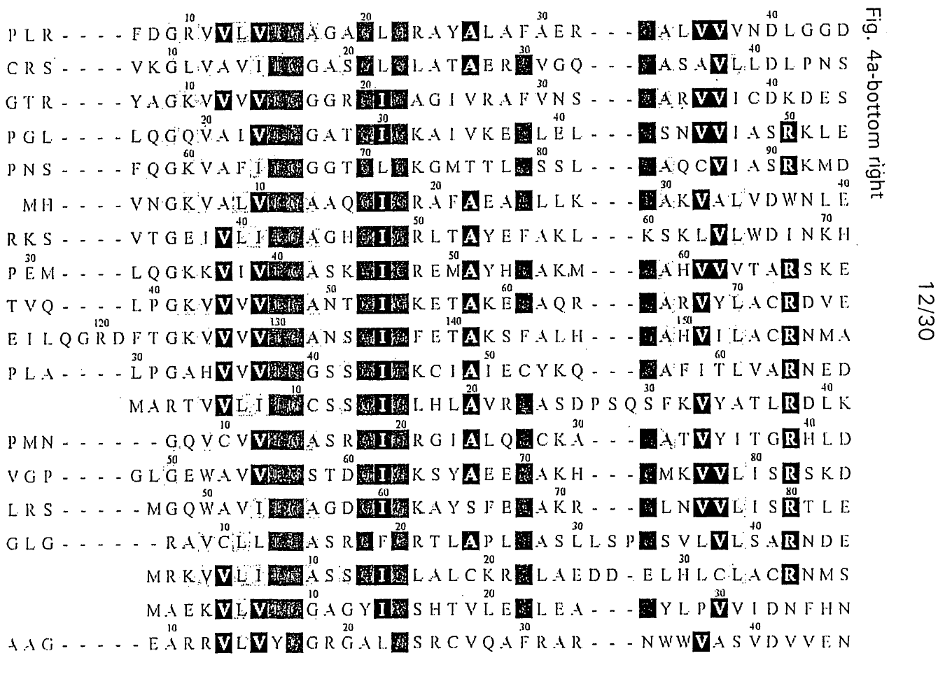

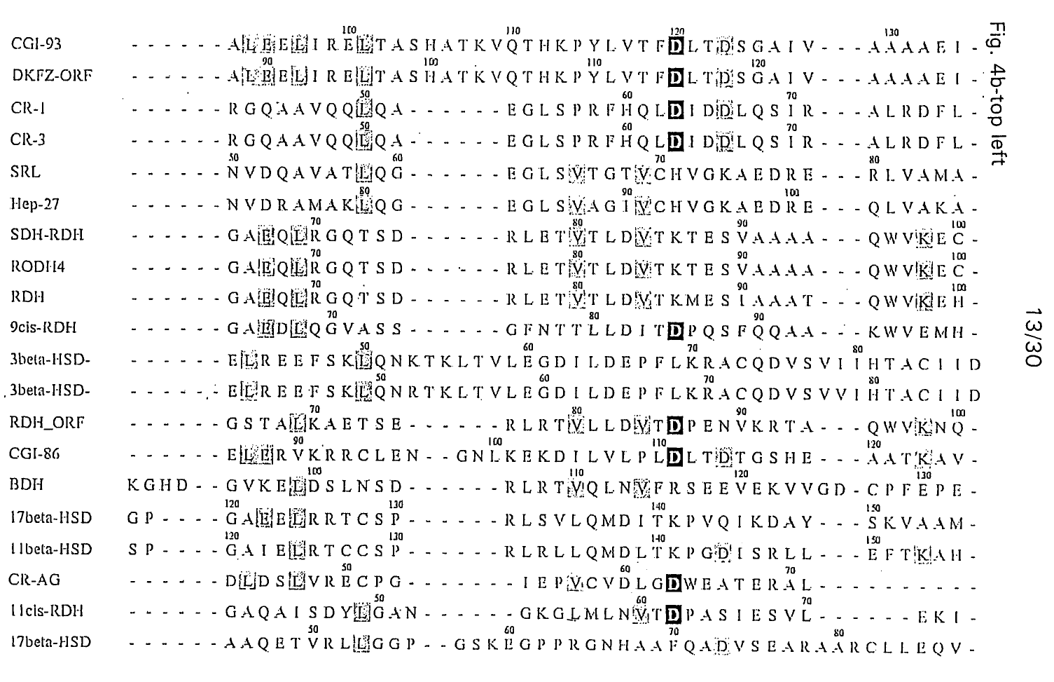

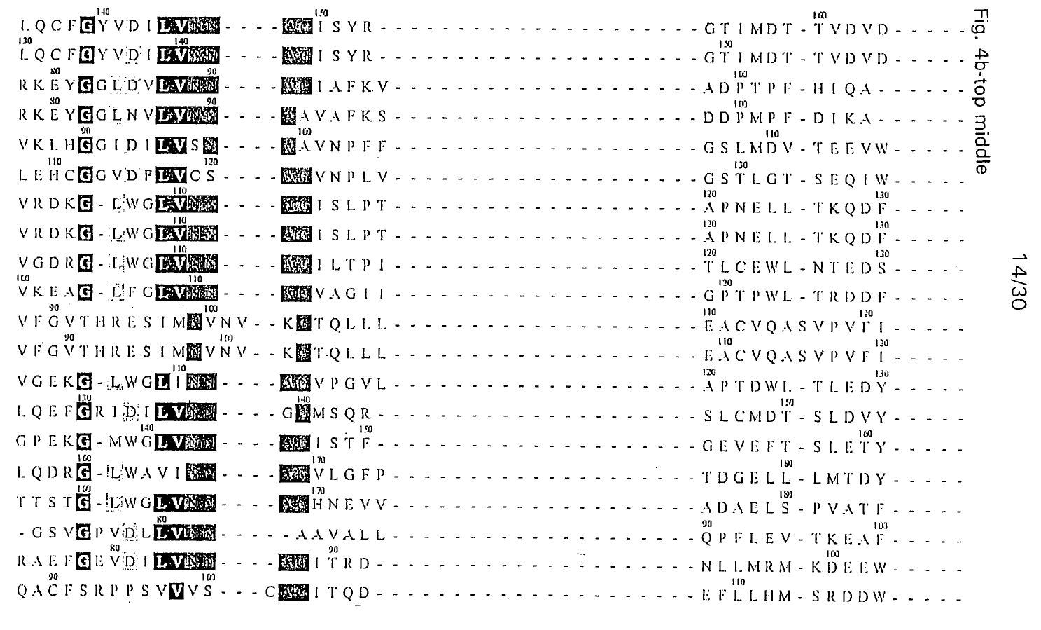

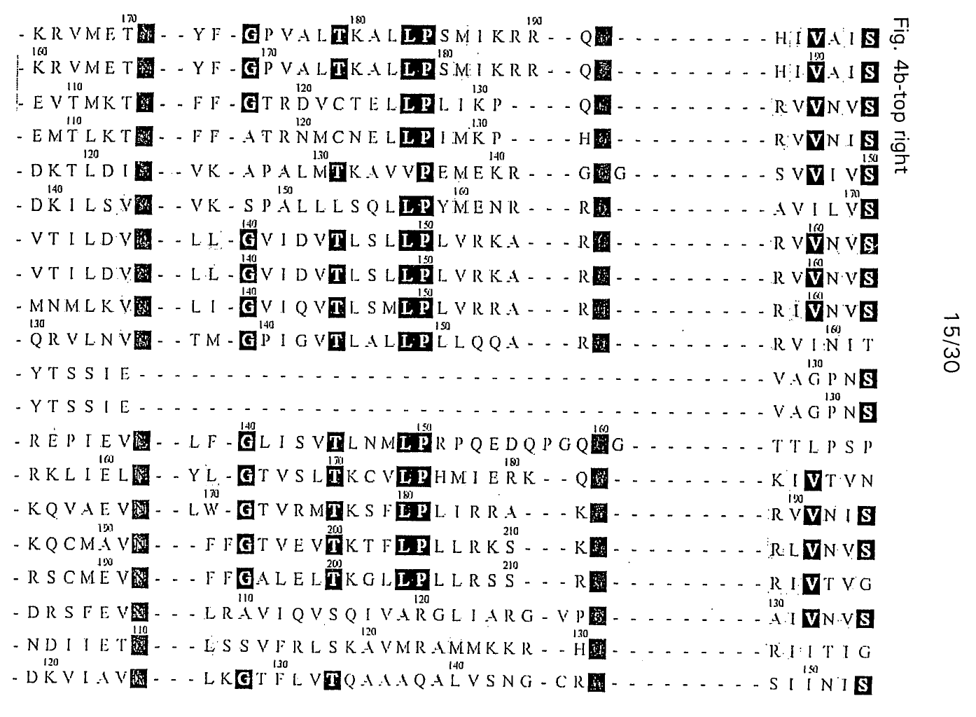

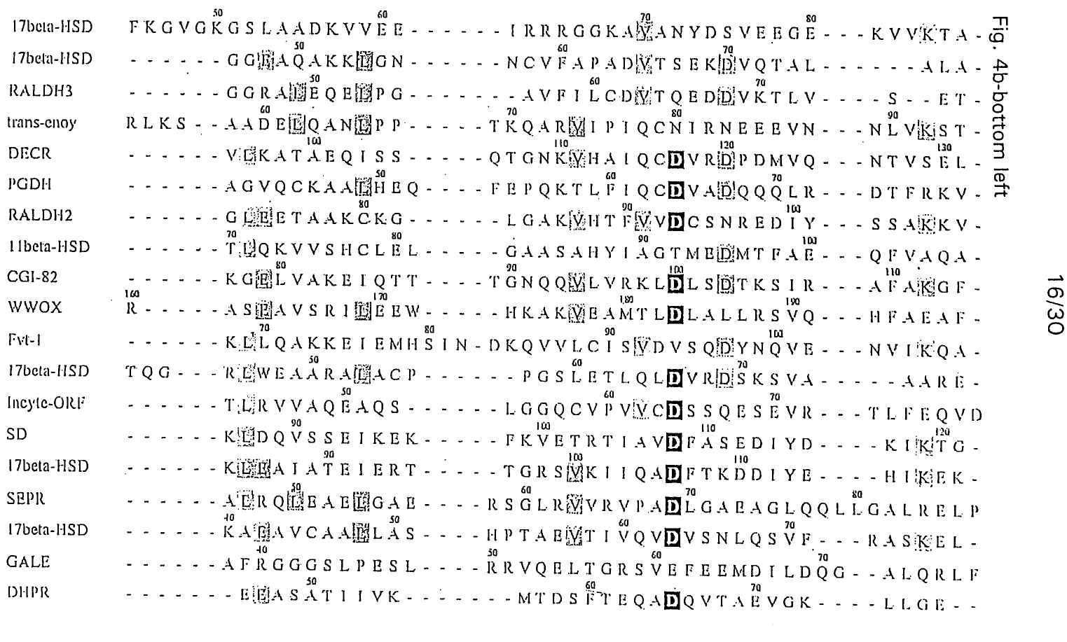

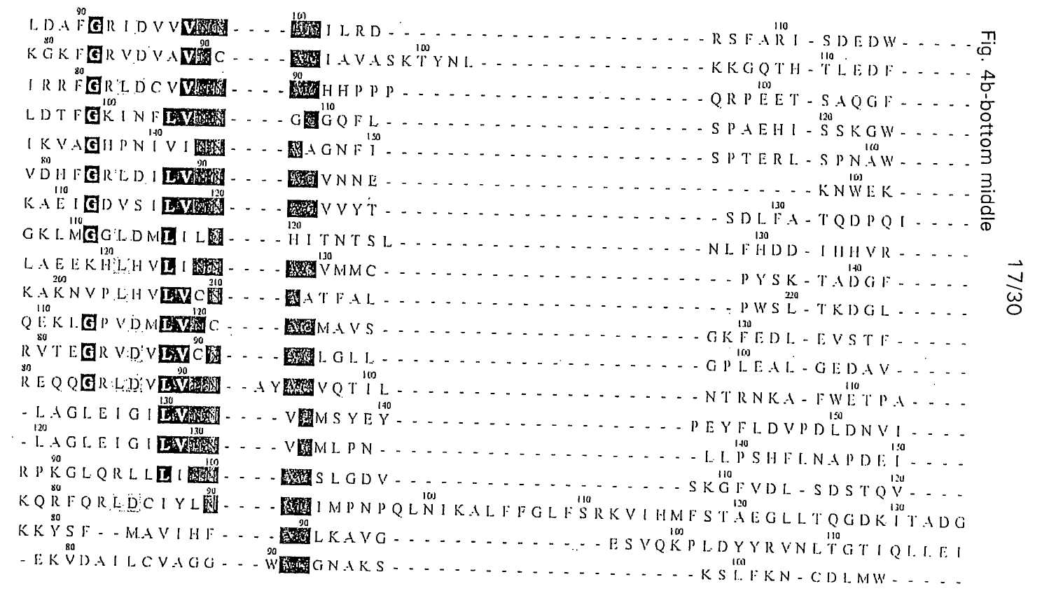

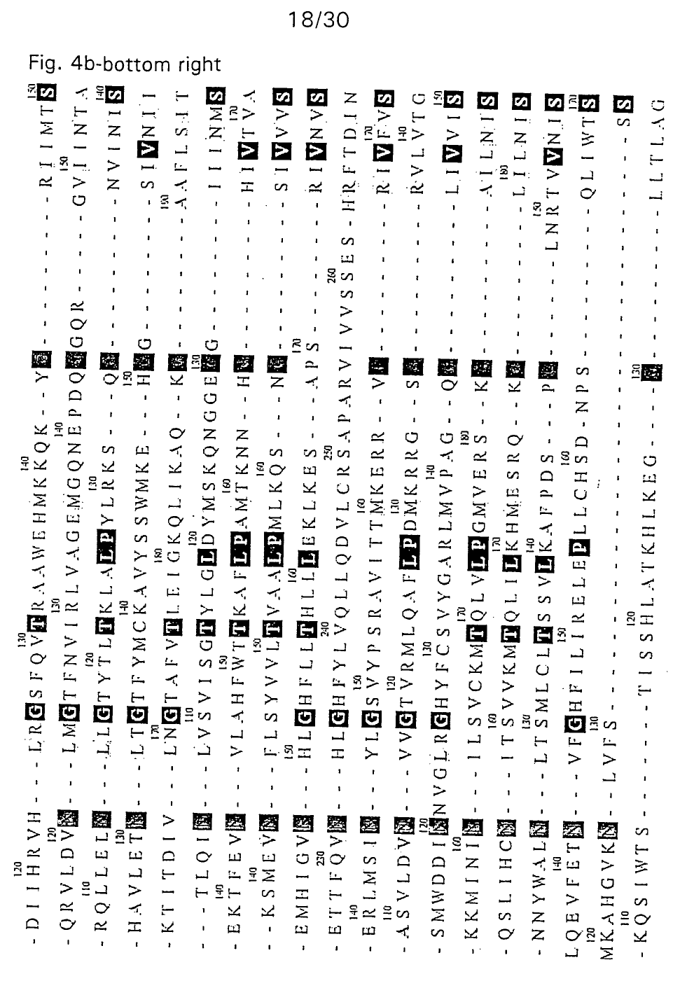

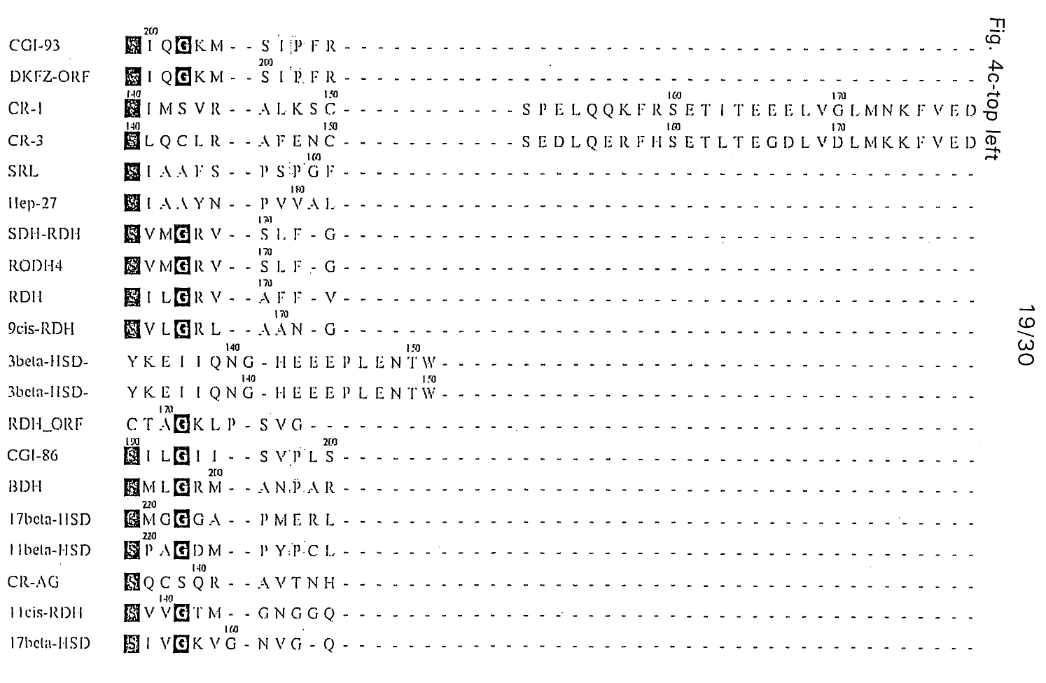

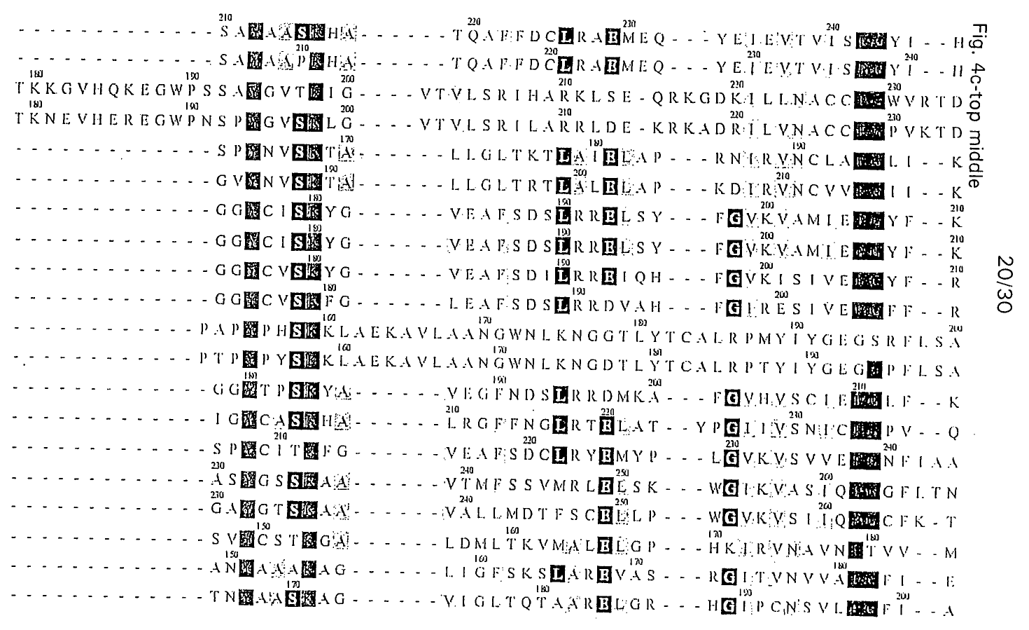

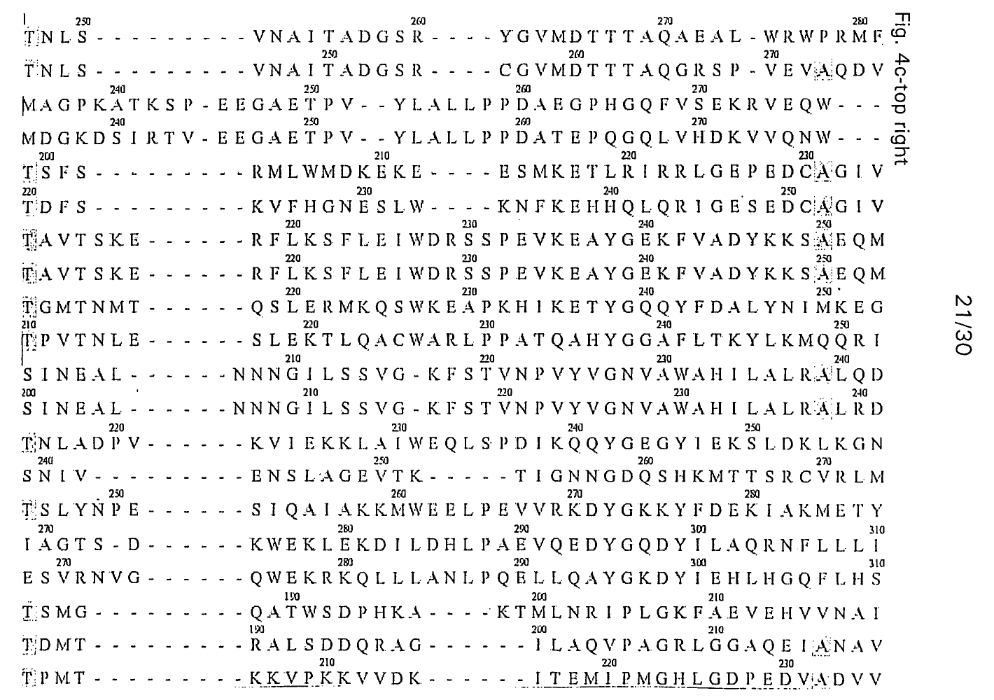

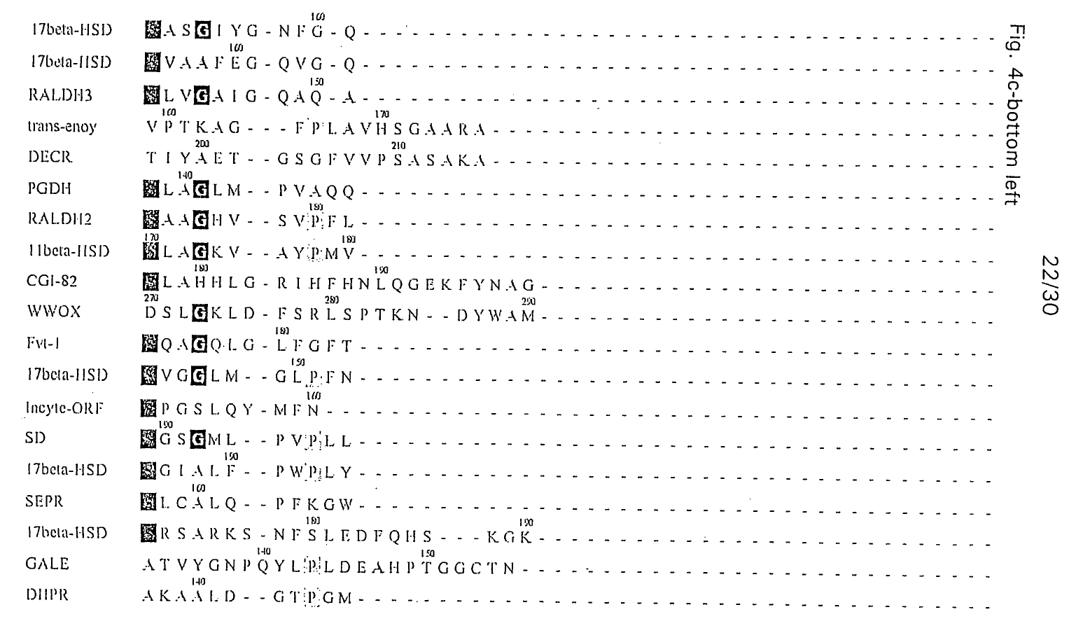

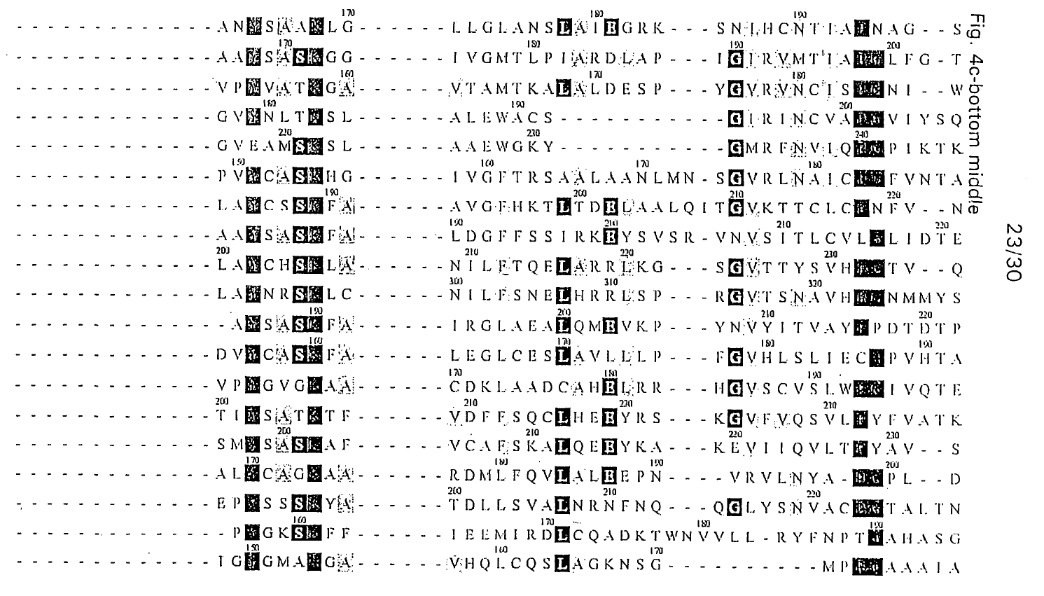

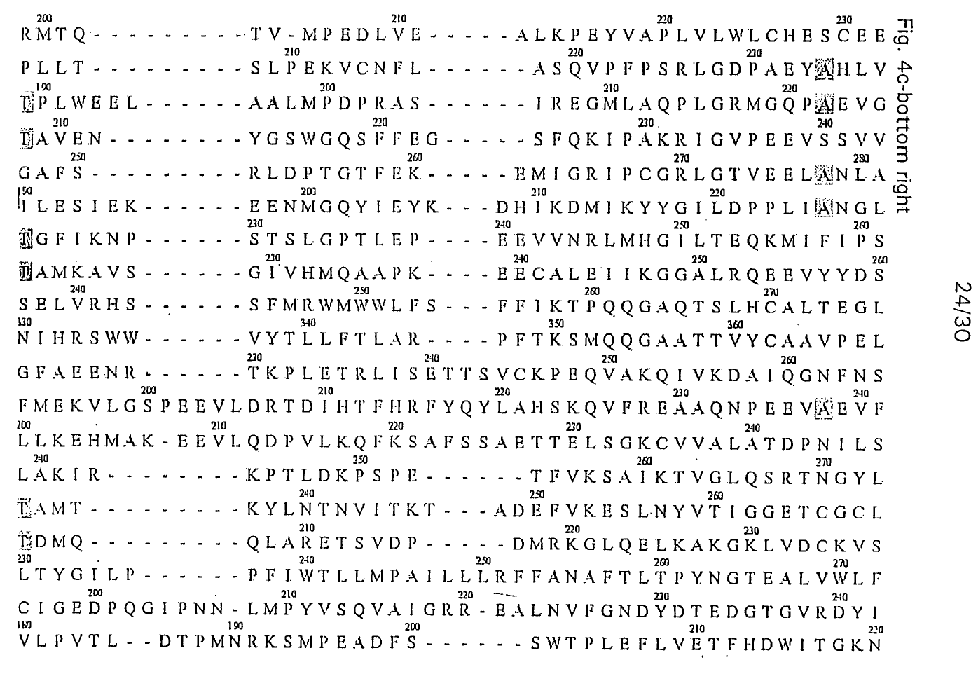

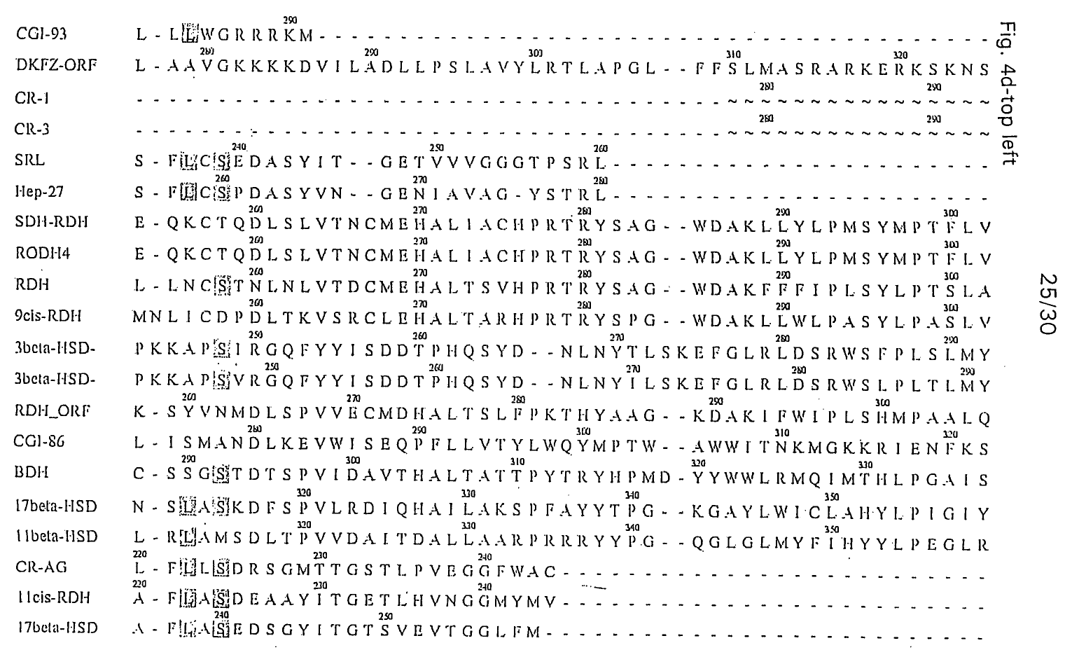

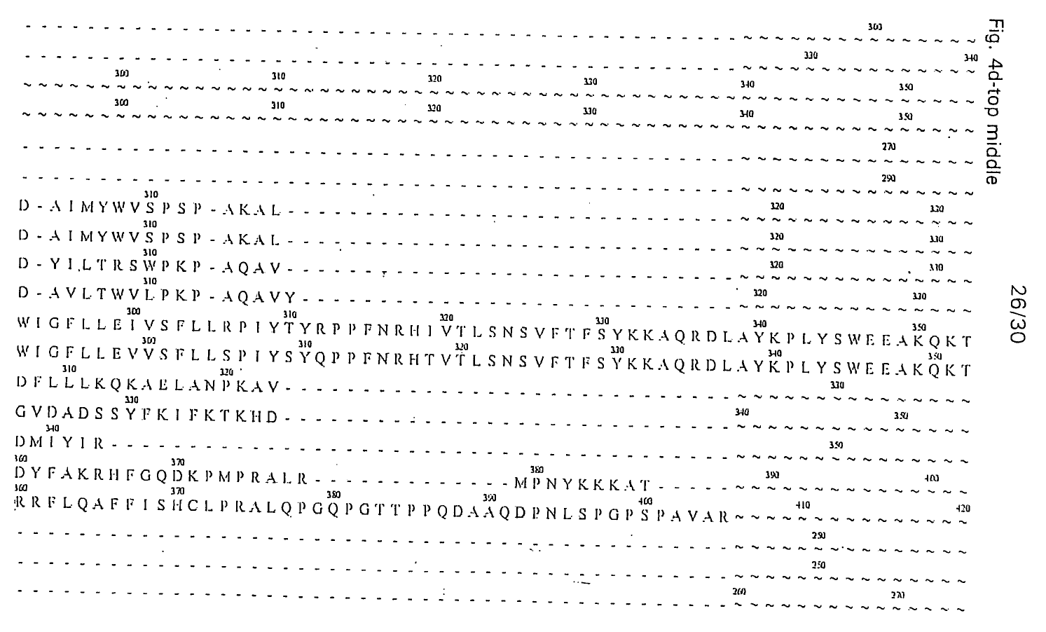

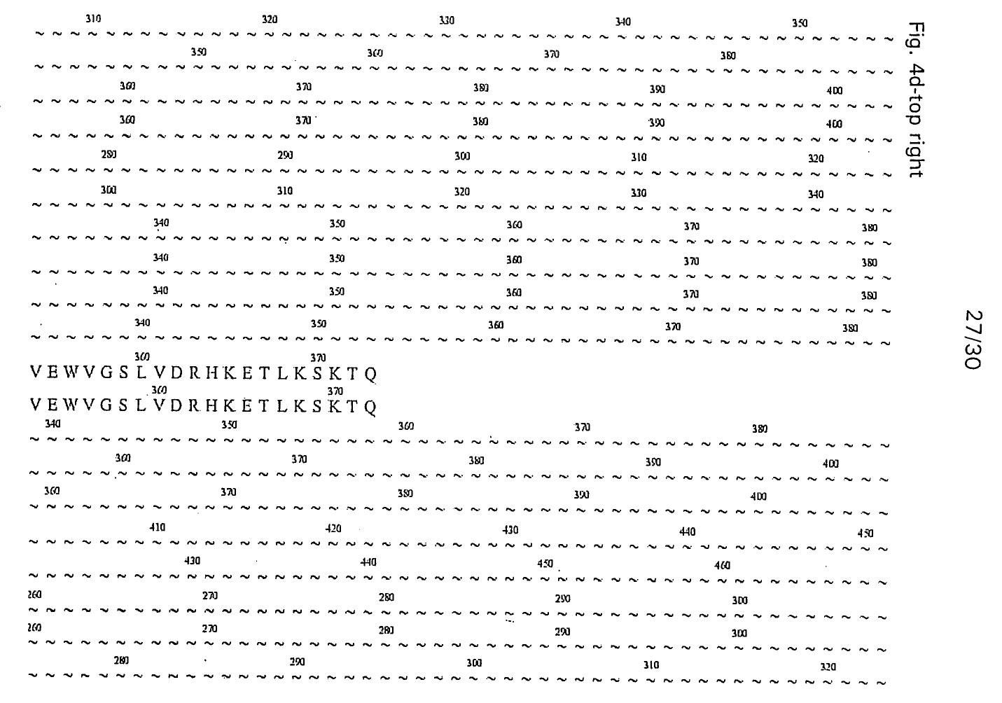

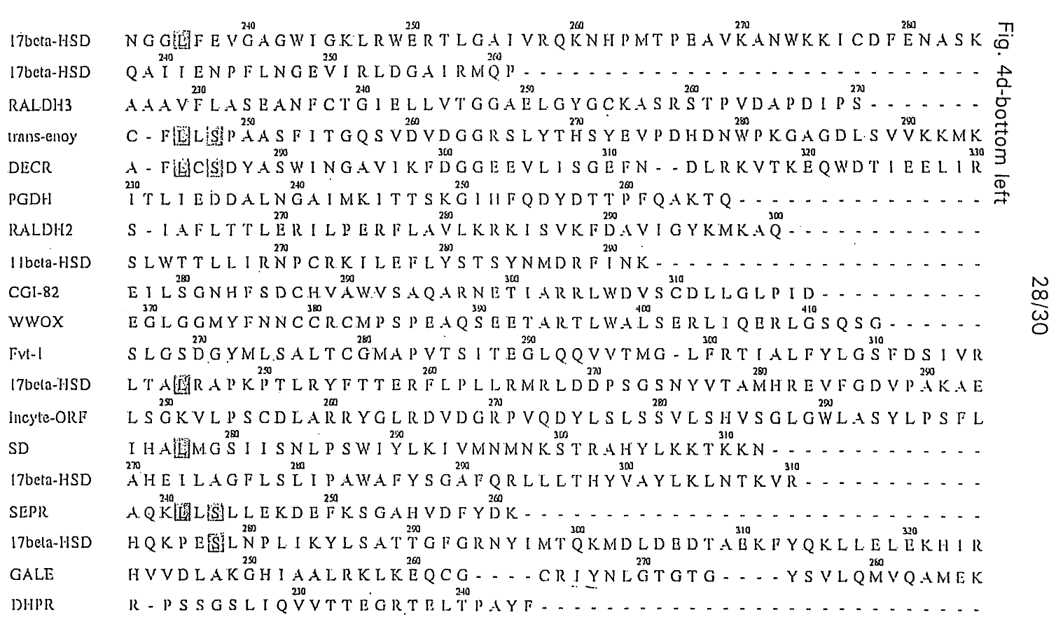

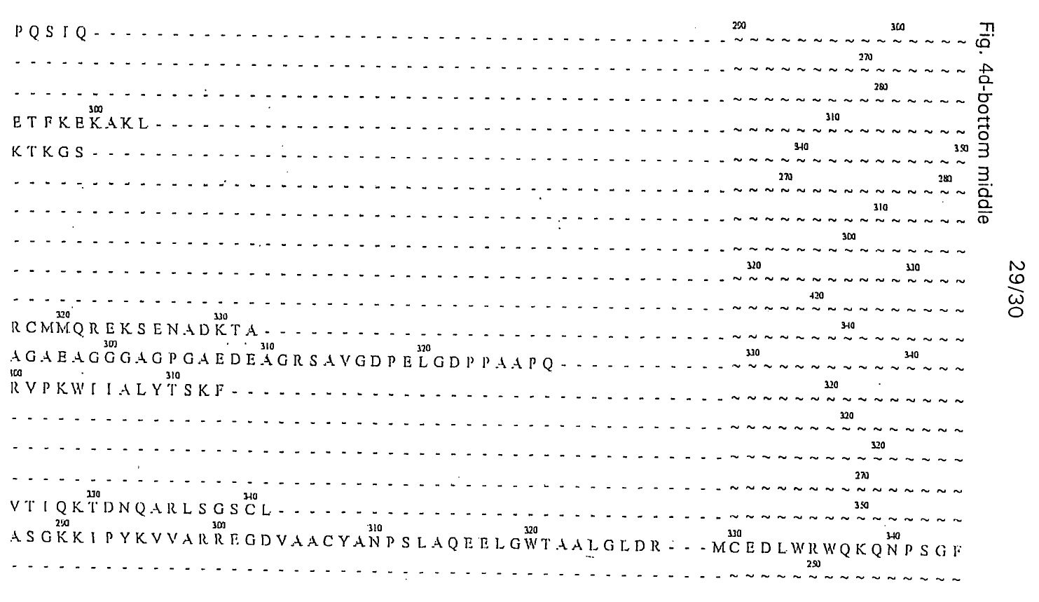

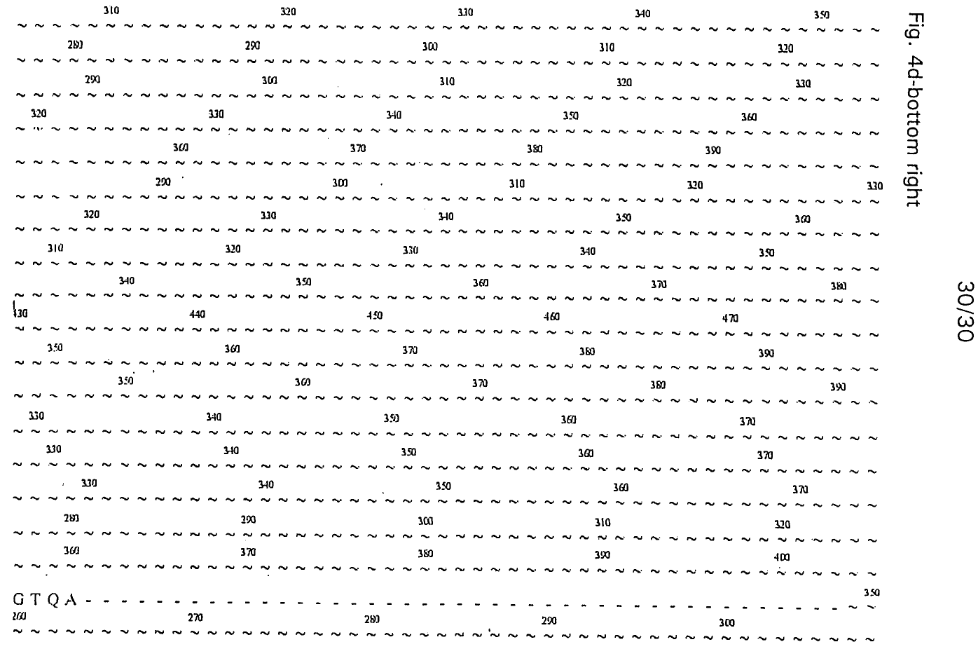

FIG. 4 shows an alignment of human SDRs. 39 human SDR proteins were found in a database using the algorithm according to the invention. Throughout the various SDR proteins highly conserved amino acids are underlaid in grey. As can be seen from this figure the motifs selected for the algorithm of the invention are present in most of the human SDRs.

Tab. 1 Table 1 shows human and/or vertebrate SDRs detection with the algorithm according to the invention. The detected SDRs are also subject matter of this invention. Further, Table 1 includes an EST search for each SDR detected, with which the corresponding function and localization in tissue can be found or localized.

Tab.2 Table 2 shows mouse SDRs detected with the method according to the invention and the results of EST searches by using these mouse SDRs in human tissue. Thus using SDRs of various species, e.g. mammals, allows for localization and identification of new SDRs, in particular human SDRs on a genomic level. A preselection and/or identification of the SDR employed can be performed with the method according to the invention.

Tab. 3 Table 3 shows in bacterial SDRs which were detected with the method according to the invention. Such bacterial SDRs are particularly suitable for the development of novel antibiotics.

Tab. 4 Table 4 shows FabG_ proteins, i.e. an SDR subgroup. It is possible with the method according to the invention specifically identify desired subgroups by selection of further criteria in a second search step.

Tab. 5 Table 5 shows SDRs from different fungi.

1. A method for identifying or verifying members of the short chain dehydrogenase (SDR) family comprising the steps
(a) providing a target sequence of molecules to be classified,
(b) comparing said target sequence with core SDR motifs selected from
(i) MV1 being derived from the motif MT1:TGxxxGxG by replacement of 0 to 2 amino acids,
(ii) MT2:NN(0-2:x)AG,
(iii) MT3:N, located at a position 90-110 relative to MT1,
(iv) MV4 being derived from the motif MT4:S(11-52:x)YxxxK by replacement of 0-2 amino acids and
(v) MT5:PG,
(c) determining positive SDR candidates containing
(i) at least the core SDR motifs MV1 and MV4 and
(ii) at least 7 of the 14 amino acids contained in the motifs MT1, MT2, MT3, MT4 and MT5 and
(d) classifying positive SDR candidates as belonging to the SDR family.

2. The method according to claim 1, further comprising a step
(e) ranking of the positive SDR candidates obtained according to the number of amino acids matching with motifs MT1, MT2, MT3, MT4 and MT5.

3. The method according to claim 1, wherein in step (b) the target sequence is compared with core SDR motifs selected from
(i) MT1:TGxxxGxG,
(ii) MT2:NN(0-2:x)AG,
(iii) MT3:N, located at position 90-110 relative to MT1,
(iv) MT4:S(11-52:x)YxxxK and
(v) MT5:PG,
and wherein in step (c) positive SDR candidates are determined containing
(i) at least the core SDR motifs MT1 and MT4 and
(ii) at least 7 of the 14 amino acids contained in the motifs MT1, MT2, MT3, MT4 and MT5.

4. The method according to claim 1, wherein in step (c) positive SDR candidates are determined containing
(i) at least the core SDR motifs MV1, MV4 and one of MT2, MT3 and MT5 and
(ii) at least 7 of the 14 amino acids contained in the motifs MT1, MT2, MT3, MT4 and MT5.

5. The method according to claim 1, wherein in step (c) positive SDR candidates are determined containing
(i) the core SDR motifs MV1, MV4, MT2 and MT3 or MV1, MV4, MT2 and MT5 or MV1, MV4, MT2, MT3 and MT5.

6. The method according to claim 1, wherein positive SDR candidates are determined containing the core SDR motifs MV1, MV4, MT2, MT3 and MT5.

7. The method according to claim 1, wherein in step (c) positive candidates are determined containing at least 9 of the 14 amino acids contained in the motifs MT1, MT2, MT3, MT4, and MT5.

8. The method according to claim 1, wherein MT2 is defined as NNAG.

9. The method according to claim 1, wherein MV4 is derived from the motif MT′4:S(11-52:x)YxASK by replacement of 0-2 amino acids.

10. The method according to claim 9, wherein in step (c) positive candidates are determined containing at least 9 of the 16 amino acids contained in the core motifs used.

11. The method according to claim 1, wherein MT2 and/or MT5 are extended for identifying or verifying FabG_SDRs, wherein MT_{y}2:VxVNNAG, wherein V can be replaced and MT_{y}5:PGFI, wherein F and/or I are used as search motif.

12. The method according to claim 1, further comprising one or more of the following further steps:
(i) three-dimensional structure comparison and
(ii) biological function analysis.

13. A member of the short-chain dehydrogenase (SDR) family identified with the method according to claim 1.

14. The SDR according to claim 13, wherein it is selected from the SDRs shown in Tables 1-5.

15. A method for providing modulators for members of the short chain dehydrogenase (SDR) family comprising the steps
(a) providing one or more target sequences of members of the short chain dehydrogenase family based on an algorithm using core SDR motifs for searching members of the SDR family and
(b) providing modulators, which enhance or inhibit the activity of the members of the short chain dehydrogenase family.

16. The method according to claim 15, wherein step (a) comprises the steps
(a) providing a target sequence of molecules to be classified,
(b) comparing said target sequence with core SDR motifs selected from
(i) MV1 being derived from the motif MT1:TGxxxGxG by replacement of 0 to 2 amino acids,
(ii) MT2:NN(0-2:x)AG,
(iii) MT3:N, located at a position 90-110 relative to MT1,
(iv) Mv4 being derived from the motif MT4:S(11-52:x)YxxxK by replacement of 0-2 amino acids and
(v) MT5:PG,
(c) determining positive SDR candidates containing
(i) at least the core SDR motifs MV1 and MV4 and
(ii) at least 7 of the 14 amino acids contained in the motifs MT1, MT2, MT3, MT4 and MT5 and
(d) classifying positive SDR candidates as belonging to the SDR family.

17. The method according to claim 15, wherein in step (b) a protein sequence alignment with known SDR sequences is performed for pre-selecting possible modulators.

18. A method for evaluation of lead-candidates for possible modulators of a member of the SDR family comprising the steps
(a) providing one or more target sequences of members of the short chain dehydrogenase family based on an algorithm using core SDR motifs for searching members of the SDR family,
(b) ranking the target sequences according to the number of amino acids matching with the core SDR motifs used and
(c) deriving lead-candidates from metabolites of evolutionary related SDR enzymes.

19. The method according to claim 18, wherein step (a) comprises the steps
(a) providing a target sequence of molecules to be classified,
(b) comparing said target sequence with core SDR motifs selected from
(i) MV1 being derived from the motif MT1:TGxxxGxG by replacement of 0 to 2 amino acids,
(ii) MT2:NN(0-2:x)AG,
(iii) MT3:N, located at a position 90-110 relative to MT1,
(iv) MV4 being derived from the motif MT4:S(11-52:x)YxxxK by replacement of 0-2 amino acids and
(v) MT5:PG,
(c) determining positive SDR candidates containing
(i) at least the core SDR motifs MV 1 and MV4 and
(ii) at least 7 of the 14 amino acids contained in the motifs MT1, MT2, MT3, MT4 and MT5 and
(d) classifying positive SDR candidates as belonging to the SDR family.

20. A method for providing a pharmaceutical agent comprising the steps
(a) providing tone or more target sequences of members of the short chain dehydrogenase family based on an algorithm using core SDR motifs for searching members of the SDR family,
(b) providing modulators, which enhance or inhibit the activity of the members of the short chain dehydrogenase family and
(c) formulating said modulators as pharmaceutical agent.

21. The method according to claim 20, wherein step (a) comprises the steps
(a) providing a target sequence of molecules to be classified,
(b) comparing said target sequence with core SDR motifs selected from
(i) MV1 being derived from the motif MT1:TGxxxGxG by replacement of 0 to 2 amino acids,
(ii) MT2:NN(0-2:x)AG,
(iii) MT3:N, located at a position 90-110 relative to MT1,
(iv) MV4 being derived from the motif MT4:S(11-52:x)YxxxK by replacement of 0-2 amino acids and
(v) MT5:PG,
(c) determining positive SDR candidates containing
(i) at least the core SDR motifs MV1 and MV4 and
(ii) at least 7 of the 14 amino acids contained in the motifs MT1, MT2, MT3, MT4 and MT5 and
(d) classifying positive SDR candidates as belonging to the SDR family.

22. The method according to claim 20, wherein step (b) comprises the steps
(a) providing one or more target sequences of members of the short chain dehydrogenase family based on an algorithm using core SDR motifs for searching members of the SDR family and
(b) providing modulators, which enhance or inhibit the activity of the members of the short chain dehydrogenase family.

23. The method according to claim 20, wherein a modulator is provided, which enhances the activity of the members of the short chain dehydrogenase family.

24. The method according to claim 20, wherein a modulator is provided, which inhibits the activity of the members of the short chain dehydrogenase family.

25. The method according to claim 20, wherein the validation of a modulator or a function of a SDR enzyme found with an algorithm using core SDR motifs is performed with biochemical methods.

26. The method according to claim 20, wherein expressed sequence tags and gene sequence comparison are used to provide a function of the member of the short chain dehydrogenase family, which has been identified or verified with an algorithm using core SDR motifs.

27. The method according to claim 20, wherein a modulator or a function of an SDR enzyme found with an algorithm using core SDR motifs is validated high throughput function screening for function identification, UHTS for lead compounds, molecular homology modelling, substrate docking simulations, tissue expression, cDNA arrays or analysis of disease in animal or in vitro model systems.

28. The method according to claim 20, wherein a human SDR enzyme is provided and the pharmaceutical agent is applied for therapeutic or diagnostic purposes.

29. The method according to claim 28, wherein the human SDR enzyme is selected from the human SDRs shown in Table 1 or 2.

30. The method according to claim 20, wherein an SDR from a pathogen and/or a fungi is provided to obtain a high specific pharmaceutical agent.

31. The method according to claim 30, wherein the SDR is selected from the SDRs shown in Table 3, 4 or 5.

32. The method according to claim 20, wherein an SDR enzyme with high homology is provided, which constitutes an essential enzyme.

33. The method according to claim 20, wherein an SDR enzyme with low homology or high divergence between different species is provided, which allows for a species specific modulation.

34. A pharmaceutical agent obtainable by a method according to claim 20.

35. The pharmaceutical agent according to claim 34 for the prophylaxis, treatment and/or diagnosis of diseases.

36. The pharmaceutical agent according to claim 34, which is a fungicide or antibiotic.

37. A method for detection of clinically relevant polymorphisms or single nucleotide polymorphisms comprising the steps
(a) providing one or more target sequences or members of the short chain dehydrogenase family based on an algorithm using core SDR motifs for searching members of the SDR family,
(b) ranking the members of the short chain dehydrogenase family according to the number of amino acids matching with the core SDR motifs applied, and
(c) comparing evolutionary patterns within the SDR enzymes.

38. The method according to claim 37, wherein disease mechanisms are characterised;

39. The method according to claim 37, wherein metabolisms of xenobiotics are characterised.

40. The method according to claim 37, wherein structure-function relationships are identified and/or substrates of SDR members with unknown function are identified.

41. The method according to claim 20, wherein a pharmaceutical agent for affecting immune regulation is provided by developing a modulator for 17β HSD type 3, 17β HSD type 7, 17β HSD type 8, 17β HSD type 10, 11β HSD-1, CR1, UDP glucose epimerase, SDR_SRL, AF067174, AF151840, AF151844, AF0078850, Fvt-1, HEP-27, DKFZ_ORF, WWOX_ORF, or CR3.

42. The method according to claim 20, wherein a pharmaceutical agent for affecting autoimmunity is provided by developing a modulator for 17β HSD-3, 17β HSD-8, 11β HSD-1, AF057034, U89717, CR1, AF0078850, HEP-27, or CR-3.

43. The method according to claim 20, wherein a pharmaceutical agent for wound healing or partial recovery is provided by developing a modulator for 17β HSD-3, 17β HSD-8, 11β HSD-1, U89717, CR1, AF0078850, HEP-27, or CR-3.

44. The method according to claim 20, wherein a pharmaceutical agent for treatment of leukemia is provided by developing modulators for 17-β HSD-10 or Fvt-1.

45. The method according to claim 20, wherein a pharmaceutical agent for apoptosis regulation is provided by developing a modulator for 17β HSD-10, U89717, SDR_SRL; or for providing a pharmaceutical agent for affecting immune response by providing a modulator for AF016509, or providing a pharmaceutical agent for the treatment of cancer by providing modulators for AF016509, or providing a pharmaceutical agent for affecting cell growth by providing a modulator for U89717, or providing a pharmaceutical agent for the treatment of lung carcinoma by providing a modulator for SDR_SRL, or providing a pharmaceutical agent for the regulation of inflammation or vasculitis by providing a modulator for DKFZ_ORF.
